(19) United States
(12) Patent Application Publication
(10) Pub. No.: US 2006/0024691 A1
(43) Pub. Date: Feb. 2, 2006

(54) Novel pathways in the etiology of cancer

(75) Inventor: Christopher C. Benz, Novato, CA (US)

Correspondence Address: QUINE INTELLECTUAL PROPERTY LAW GROUP, P.C., P O BOX 458, ALAMEDA, CA, 94501, US

(73) Assignee: Buck Institute for Age Research

(21) Appl. No.: 11/090,546

(22) Filed: Mar. 24, 2005

Related U.S. Application Data
(60) Provisional application No. 60/556,774, filed on Mar. 25, 2004.
(60) Provisional application No. 60/580,534, filed on Jun. 16, 2004.
(60) Provisional application No. 60/629,691, filed on Nov. 19, 2004.

(51) Int. Cl. A61K 31/454; A61K 31/138; C12Q 1/68; G01N 33/574

(52) U.S. Cl. 435/6; 435/7.23; 514/321; 514/651

(57) ABSTRACT

This invention pertains to the identification of two novel epithelial signaling pathways in ER-positive breast cancer s and the discovery that the cellular biology and (likely also the clinical outcome) of ER-positive breast cancer cells is unexpectedly altered when these signaling pathways are activated. The first pathway pertains to the discovery that NF-κB activation and/or DNA binding is implicated in the etiology of ER-positive breast (and other) cancers. The second pathway involves ligand-independent quinine-mediated ER activation by posphorylation (e.g. on SER-118 and SER-167 residues of ER) and nuclear translocation of full-length (67 kDA) ER as well as the phorphorylating activation of a truncated and nuclear-localized ER variant (˜52 kDa).

### CROSS-REFERENCE TO RELATED APPLICATIONS

This application claims benefit of and priority to U.S. Ser. No. 60,556,774, filed on Mar. 25, 2004, U.S. Ser. No. 60/580,534, filed on Jun. 16, 2004, and 60/629,691, filed on Nov. 19, 2004, all of which are incorporated herein by reference in their entirety for all purposes.

### STATEMENT AS TO RIGHTS TO INVENTIONS MADE UNDER FEDERALLY SPONSORED RESEARCH AND DEVELOPMENT

This work was sponsored by NIH/NCI Grant Nos: CA71468 and AG020521. The Government of the United States of American may have certain rights in this invention.

### FIELD OF THE INVENTION

This invention pertains to the field of cancer and cancer therapeutics. In particular two novel pathways are identified as implicated in the etiology of various cancers. This provides novel targets for diagnosis, prognosis, and intervention.

### BACKGROUND OF THE INVENTION

Like other members of the steroid receptor superfamily, the ER˜ protein has a modular domain structure with an N-terminal activation function (AF-1) region, a DNA binding domain (DBD), a ligand binding domain (LDB), and a Cterminal activation function (AF-2) region (1). The classical ligand (estrogen) binding stimulation of ER˜ induces structural reorganization of the LBD, enabling recruitment of coactivators to the AF-2 region (2,3). Additionally, a growing literature has documented ligand-independent mechanisms for ER˜ activation by processes thought to target the AF-1 region, initiated by various membrane localized receptors and transduced by mitogen activated protein kinase (MAPK) phosphorylaton of key AF-1 serine (Ser) residues, principally Ser-118 but also Ser-167 (4-8). Interestingly, ligand activation of ER˜ also results in Ser-118 phosphorylation although the kinase responsible remains controversial (9, 10).

Another level of complexity in understanding ERα function has been the large number of reports documenting cell expression of alternately-spliced ERα transcripts (11-20). These occur as single or multiple exonic deletions that may involve almost any of the eight genomic exons that encode full-length ERα except the first which is needed to initiate translation and the last which encodes the final C-terminal epitopes of the full-length receptor as well as the regulatory 3′ untranslated region (21). While protein evidence for endogenous expression of these splice variants is scant, all of the exon-deleted splice variants detected and identified by sequencing of RT-PCR products are predicted to encode receptor proteins lacking function and epitopes specific to their missing domain sequences. Of note, except for the deletion of exons 3 or4, these ER˜ single exon splice variants are predicted to lack all C-terminal receptor epitopes as a result of prematurely terminated translation caused by frame-shifting, exemplified by the well studied exon-5 deleted and exon-7 deleted variants (11, 23, 24).

### SUMMARY OF THE INVENTION

This invention pertains to the identification of two novel epithelial signaling pathways in ER-positive breast cancer s and the discovery that the cellular biology and (likely also the clinical outcome) of ER-positive breast cancer cells is unexpectedly altered when these signaling pathways are activated.

The first pathway pertains to the discovery that NF-κB activation and/or DNA binding is implicated in the etiology of ER-positive breast (and other) cancers. The second pathway involves ligand-independent quinine-mediated ER activation by phosphorylation (e.g. on SER-118 and SER-167 residues of ER) and nuclear translocation of full-length (67 kDA) ER as well as the phorphorylating activation of a truncated and nuclear-localized ER variant (˜52 kDa). These pathways provide convenient targets for intervention and/or for diagnosis/prognosis of various cancers.

Thus in one embodiment, this invention provides a method of identifying cancer patients less likely to respond to hormonal therapy (e.g., for whom hormonal therapy is contraindicated). The method typically involves obtaining a biological sample from a cancer patient where the biological sample comprises cancer cells; and determining NF-κB levels, activity or DNA binding where a patient having higher NFκB levels, activity or DNA binding, as compared to the NF-κB levels, activity or DNA binding found in a normal healthy subject indicates that the patient is less likely to respond to hormonal therapy. In various embodiments the cancer patient is a cancer patient having ER positive breast cancer. In certain embodiments the determining comprises determining NF-κB DNA binding. In certain embodiments the determining comprises determining NF-κB activation.

This invention also provides a method of evaluating the prognosis of a patient having breast cancer. The method typically involves obtaining a biological sample from the cancer patient where the biological sample comprises cancer cells; and determining NFκB levels, activity or DNA binding in the cancer cell(s) wherein higher NFκB levels, activity or DNA binding, as compared to the levels, the activity, or the DNA binding found in a normal healthy subject is an indicator of a higher risk of cancer recurrence or relapse. In certain embodiments the cancer patient is a cancer patient having ER positive breast cancer.

Also provided is a method of mitigating one or more symptoms of breast cancer in a subject having ER-positive breast cancer. The method typically involves administering to the patient a NF-κB inhibitor. In certain embodiments the inhibitor inhibits NF-κB expression. In certain embodiments the inhibitor inhibits DNA binding by NF-κB. In certain embodiments the inhibitor is selected from an the group consisting of an inhibitor listed in Table 1, an inhibitor listed in Table 2, an inhibitor listed in Table 3, and an inhibitor listed in Table 4.

In certain embodiments this invention also provides a method of mitigating one or more symptoms of a cancer associated with activation of a steroid nuclear receptor. The method typically involves administering to the patient an agent selected from the group consisting of a MAPK inhibitor, and an inhibitor of the vitamin K cycle. In certain embodiments the cancer is breast cancer, a prostate cancer, and/or an ovarian cancer. In certain embodiments the agent is an inhibitor of the vitamin K cycle. In certain embodiments the agent is an inhibitor of vitamin K3 production. In certain embodiments the agent is a vitamin K analogue. In certain embodiments the agent is a MAPK inhibitor. In various embodiments the agent is selected from the group consisting of UO126, CNI-1493, SB-242235, PD 98059, ALX-385-008 (Apigenin), ALX-270-328 (2-(4-Chlorophenyl)-4-(fluorophenyl)-5-pyridin-4-yl-1,2-dihydropyrazol-3-one), ALX-350-290 (Debromohymenialdisine), ALX-350-289 (10Z-Hymenialdisine), LKT-H9861 (Hypericin), ALX-350-030 (Hypericin native), ALX-350-258 (Parthenolide), ALX-385-023 (PD 98,059), ALX-270-258 (PD 169,316), ALX-270-324 (Raf1 Kinase Inhibitor I), ALX-270-259 (SB202190), ALX-270-268 (SB202190 hydrochloride), ALX-270-179 (SB203580), ALX-270-325 (SB220025), ALX-270-351 (SB239063), ALX-270-257 (SC68376), ALX-270-260 (SKF-86002), ALX-270-237 (U0126), and ALX-270-336 (ZM 336372).

In certain embodiments this invention provides methods of inhibiting activation of a nuclear receptor in the steroid receptor family in a cell. The methods typically involves contacting the cell with an agent selected from the group consisting of a MAPK inhibitor, and an inhibitor of the vitamin K cycle. In certain embodiments the cell is a cancer cell (e.g., a breast cancer cell). In certain embodiments the receptor is selected from the group consisting of an estrogen receptor (ER), a testosterone receptor, a glucocorticoid receptor, and a progesterone receptor.

Also provided is a method of mitigating a cancer characterized by ligand-independent activation of a nuclear steroid receptor. The method typically involves inhibiting a NQOR1 pathway and/or a MAPK pathway in cells comprising the cancer. In various embodiments the cancer is selected from the group consisting of ER-positive breast cancer, uterine cancer, and prostate cancer.

In various embodiments this invention also provides a method of identifying ligand-independent activation an estrogen receptor in a cell. The method typically involves detecting the 52 kDa variant of the estrogen receptor in the nucleus of the cell, where increased levels of the 52 kDa variant in the nucleus of the cell as compared to that found in a cell that is not undergoing ligand-independent activation of the estrogen receptor indicates that ligand-independent activation of an estrogen receptor is occurring in the cell. In various embodiments the detecting comprises detecting the amount of 52 kDa variant in the nucleus and/or detecting the amount of phosphorylated 52 kDa variant in the cell, and/or detecting the ratio of phosphorylated to unphosphorylated 52 kDa variant in the cell.

This invention also provides a method of selecting a therapeutic regimen for treatment of a cancer in a subject. The method typically involves providing a biological sample from the subject comprising cancer cells; detecting the 52 kDa variant of the estrogen receptor in the nucleus of the cancer cells; wherein increase levels of the 52 kDa variant in the nucleus of the cells as compared to that found in a cell that is not undergoing ligand-independent activation of the estrogen receptor indicates that the subject is a candidate for treatment of a cancer mediated by ligand-independent activation of the estrogen receptor. In certain embodiments the detecting comprises detecting the amount of phosphorylated 52 kDa variant in the cell, and/or detecting the ratio of phosphorylated to unphosphorylated 52 kDa variant in the cell. The method can further comprise treating those candidates for treatment of a cancer mediated by ligand-independent activation of the estrogen receptor comprising by inhibiting a NQOR1 pathway and/or a MAPK pathway in cells comprising the cancer.

Kits are also provided for mitigating ligand independent activation of a nuclear steroid receptor. In certain embodiments the kits comprise a container containing a MAPK inhibitor and/or an inhibitor of a vitamin K cycle; and instructional materials teaching the use of a MAPK inhibitor and/or a quinine inhibitor for reducing ligand-independent activation of a nuclear steroid receptor.

Kits are also provided for identifying ligand-independent activation of an estrogen receptor. In certain embodiments the kits comprise one or more reagents for detecting the amount and/or phosphorylation of the 52 kDa variant of the estrogen receptor in the nucleus of a cell. The kits can, optionally, further comprise instructional materials teaching the detection of the amount and/or phosphorylation of the 52 kDa variant in the nucleus of a cell as an indicator of ligand-independent activation of the estrogen receptor.

Kits are also provided for mitigating one or more symptoms of breast cancer. In certain embodiments the kits comprise an NFκB inhibitor and an antiestrogen.

Methods are provided for mitigating one or more symptoms of breast cancer. The methods typically involve identifying a breast cancer patient wherein the breast cancer is an ER-positive breast cancers with elevated NFkB activity; and administering, one or more NFkB inhibitors in conjunction with an antiestrogen. In certain embodiments the NFκB inhibitor is parthenolide, or a parthenolide analogue. In certain embodiments the antiestrogen is tamoxifen or 2-(4-Hydroxy-phenyl)-3-methyl-1-[4-(2-piperidin-1-yl-ethoxy)-benzyl]-1H-indol-5-ol hydrochloride (ERA-923). In various embodiments the NFκB inhibitor is administered before the antiestrogen, simultaneously with the antiestrogen, or after the antiestrogen.

Compositions are provided for mitigating one or more symptoms of breast cancer. In various embodiments the compositions comprise an NFκB inhibitor combined with an antiestrogen. In various embodiments the composition is formulated in a unit dosage formulation.

### DEFINITIONS

NF-κB (Nuclear Factor-kappa.B) is a eucariotic transcription factor of the rel family, which is normally located in the cytoplasm in an inactive complex. The predominant form of NF-kB is a heterodimer composed of p50 and p65 subunits, bound to inhibitory proteins of the IkB family, usually IkB-alpha (Thanos and. Maniatis (1995) Cell 80: 529-532). NF-κB is activated in response to different stimuli, among which phorbol esters, inflammatory cytokines, UV radiation, bacterial and viral infections. Stimulation triggers the release of NF-κB from IkB in consequence of the phosphorylation and the following degradation of the IkB-alpha protein (Baeuerle and Henkel (1994) Annu. Rev. Immunol. 12: 141-179). Once it is activated, NF-κB translocates to the nucleus where it binds to DNA at specific kb-sites and induces the transcription of a variety of genes encoding proteins involved in controlling the immune and inflammatory responses, among which a variety of interleukins, the tumor necrosis factor alpha, the NO synthase and the cyclo-oxigenase 2 (Grimm and Baeuerle (1993) Biochem. J. 290: 297-308). Accordingly, NF-κB has been considered an early mediator of the immune and inflammatory responses and it is involved in the control of cell proliferation and in the pathogenesis of various human diseases, among which rheumatoid arthritis (Beker et al. (1995) Clin. Exp. Immunol. 99: 325), ischemia (Salminen et al. (1995) Biochem. Biophys. Res. Comm. 212: 939), arteriosclerosis (Baldwin (1996) Annals Rev. Immunol. 14: 649), as well as in the pathogenesis of the acquired immunodeficiency syndrome (AIDS) (Lenardo and Baltimore (1989) Cell 58: 227-229).

The “in conjunction with” when used in reference to the use of two or more drugs (e.g., an NFκB inhibitor and an antiestrogen) indicates that the two drugs are administered so that there is at least some chronological overlap in their physiological activity on the organism. Thus, for example, an NFκB inhibitor and an antiestrogen can be administered simultaneously and/or sequentially. In sequential administration there may even be some substantial delay (e.g., minutes or even hours or days) before administration of the second drug as long as the first administered drug has exerted some physiological alteration on the organism that persists when the second drug is administered or becomes active in the organism.

### BRIEF DESCRIPTION OF THE DRAWINGS

FIG. 1 illustrates ER activation associated with serine phosphorylation.

FIG. 2 illustrates intracellular effects of redox-cycling quinines.

FIG. 3 illustrates the phosphorylation of ER variant and ERK1/2 induced by 4-OHE quinine.

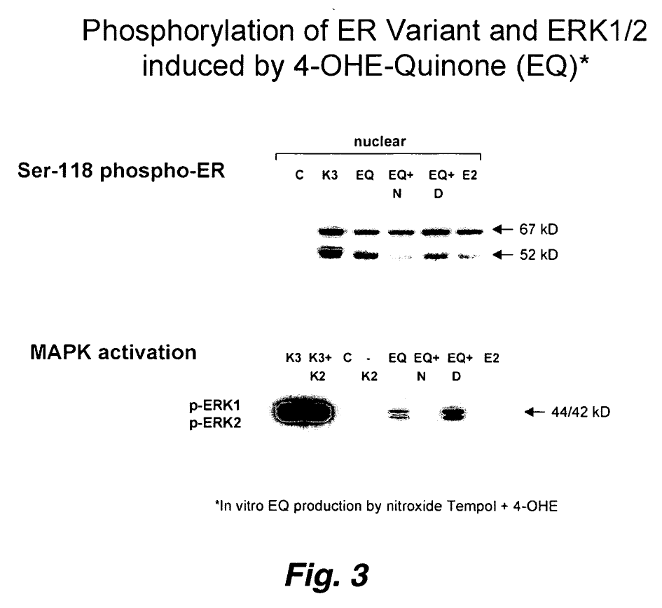

FIG. 4 shows a summary of initial results.

FIG. 5 illustrates a model for ligand-independent ER effects by redox-active quinines.

FIGS. 6A, 6B, and 6C illustrate K3 induced nuclear translocation of 67 kD ERα and identification of a nuclearly localized 52 kD ERα variant. FIG. 6A: MCF7 cells growing in charcoal stripped media were untreated (control) or treated for 30 minutes with 100 μM menadione or 10 nM estradiol. Fixed cells were immunostained with ERα antibody F-10 (ER) and nuclei visualized by Dapi. FIG. 6B: Nuclear and cytoplasmic (Cyto) extracts prepared from MCF7 cells untreated (C) or treated as above with menadione (K3) or estradiol (E) were immunoblotted with the ERα N-terminal antibody 62A3 or the ERα C-terminal antibody F-10. Molecular weight indications were determined by the mobilities of a full range of recombinant protein markers (not shown). FIG. 6C: Western blots with lanes identically loaded with nuclear extracts from MCF7 cells treated as above with K3 or E, were cut in half and probed with either F-10 or the ERα C-terminal antibody H222 (left panel), or with F-10 or the ERα C-terminal antibody D75 (right panel).

FIG. 7 shows that the 52 kD ERα nuclear variant and 67 kD ERα after menadione (K3) treatment are bound within the chromatin/nuclear matrix pellet. Nuclei from control (C) MCF7 cells or cells treated with K3 or E (as described in FIG. 6) were extracted with high-salt (0.42 M NaCl) to give a high-salt nucleoplasmic fraction and a pellet-bound chromatin/nuclear matrix fraction solubilized by DNase I and detergent treatment (Pellet). Westerns blots prepared from these two nuclear fractions as well as lysates of solubilized total nuclei (T-Nu) were probed with the exon 4 ERα antibody SRA-1000 (top panel), the C-terminal ERα antibody D75 (middle panel), and an antibody to the nucleoplasm localized ERK1/ERK2 kinases (bottom panel). Molecular weight indications were determined as described in FIG. 6.

FIGS. 8A, 8B, 8C, and 8BD illustrate RT-PCR identification, restriction enzyme analysis, and sequencing of two ERα variant transcripts containing simultaneous deletions of exons 6 and 7 and exons 5 and 6. FIG. 8A: An aliquot of an RT reaction which used oligo dT primed RNA from MCF7 cells growing in charcoal stripped media was PCR analyzed using an ERα exon 4/8 primer pair. PCR products were electrophoresed on a 8% polyacrylamide gel and visualized by ethidium bromide staining. Lane M contains ΦX HaeIII digested DNA as size markers with the fragment sizes (in base pairs, bp) indicated by arrows. Lane 1 contains 10 μl of PCR product. Lane 2 contains an aliquot of the 460 bp band obtained following gel excision of the PCR product band and its reamplification. Lane 3 contains an aliquot of the 600 bp band obtained following gel excision of the PCR product band and its reamplification. FIG. 8B: Products from the restriction enzyme digestion of the 460 bp band were electrophoresed on an 8% polyacrylamide gel and visualized by ethidium bromide staining. Lane M is identical to that described in panel A. Lane 1 contains the products from BglII digestion of the 460 bp band. Lane 2 contains the products from NcoI digestion of the 460 bp band. Lane 3 contains an undigested aliquot of the 460 bp band. FIG. 8C: Identical to the PCR shown in panel A except for the use of an ERα exon 4/7 primer pair. Lane M is as described in panel A. Lane 1 contains 10 μl of PCR product. Lane 2 contains an aliquot of the 295 bp band obtained following gel excision of the PCR band and its reamplification. Lane 3 contains an aliquot of the 424 bp band obtained following gel excision of the PCR band and its reamplification. FIG. 8D: Sequencing of the gel purified 460 bp band which remained following BglII digestion of the 460 bp product shown in lane 2 of panel A and indicating the precise joining of exon 5 with exon 8 (left panel). Sequencing of the 295 bp band shown in lane 2 of panel C and indicating the precise joining of exon 4 to exon 7 (right panel).

FIGS. 9A, 9B, and 9C show that menadione (K3) treatment of MCF7 and T47D cells induces serine-118 phosphorylation (p-Ser-118) on the 52 kD ERα nuclear variant as well as on nuclear translocated 67 kD ERα. FIG. 9A: Western blots of MCF7 nuclear extracts prepared and fractionated as described in FIG. 7 were probed with the p-Ser-118 ERα antibody 16JR (top panel) or its control counterpart 62A3, directed against the unphosphorylated Ser-118 epitope of ERα (bottom panel). Asterisks (*) in the top panel indicate a ˜56 kD artifact signal corresponding to a nucleoplasmic (and cytoplasmic) protein detected by the 16JR antibody following K3 treatment; this band in undetectable by the control 62A3 antibody or any of six other ERα antibodies. FIG. 9B: Immunoblots of total nuclear extracts prepared from control (C) or treated (K3, E) MCF7 cells that were also co-treated as indicated with the MAPK inhibitor U0126 (U, 10 μM). As shown, Western blots were probed with either the ERα p-Ser-118 antibody 16JR, the ERα C-terminal antibody F-10, a p-ERK1/p-ERK2 specific antibody, or a total ERK1/ERK2 specific antibody. The asterisk (*) in the top panel indicates the same 16JR-associated artifact signal seen after K3 treatment as described in panel A. FIG. 9C: Immunoblots of total nuclear extracts prepared from control (C) or treated (K3, K3/U, E, E/U) T47D cells as described for MCF7 cells in panel B. Western blots were probed with the indicated antibodies and show similar results including the same artifact signal (asterisk) as seen in the above panels for MCF7.

FIG. 10 Menadione (K3) treatment induces MAPK-dependent serine-167 phosphorylation (p-Ser-167) on the 52 kD ERα nuclear variant but not on nuclear translocated 67 kD ERα. Immunoblots of total nuclear extracts prepared from control (C) or treated (K3, E) MCF7 cells that were also co-treated as indicated with the MAPK inhibitor U0126 (U, 10 ∝⊂). As shown, a Western blot were first probed with an ERα p-Ser-167 specific antibody and then reprobed (without stripping) with the ERα N-terminal specific antibody 62A3.

FIG. 11 shows that N-acetyl cysteine (N, 10 mM) and dicumarol (D, 100 μM) effectively block menadione (K3)-induced Ser-118 and Ser-167 phosphorylation of ERα without inhibiting MAPK activity. Immunoblots of total nuclear extracts prepared from control (C) or treated (K3, E) MCF7 cells that were also co-treated as indicated with the arylation inhibitor N or the NQO1 inhibitor D. As shown, Western blots were probed with either the ERα p-Ser-118 specific antibody, the ERα p-Ser-167 specific antibody, a p-ERK1/p-ERK2 specific antibody, or a total ERK1/ERK2 specific antibody. Asterisks (*) indicate the same ˜56 kD artifact signal described in FIG. 9.

FIG. 12 shows primary breast cancer samples (T1, T2, T3) with Ser-118 phosphorylated 52 kD ERα nuclear variant as well as 67 kD ERα expression. Representative cryobanked primary breast cancer samples known to be ERα-positive were subjected to high-salt (0.42M NaCl) extraction, as routinely used to quantitative receptor content, and the remaining pellet solubilized with DNase I and detergent treatment (Pellet). As indicated, Western blots of the two sets of tumor fractions were probed with either the ERα p-Ser-118 specific antibody 16JR or the ERα C-terminal specific antibody F-10. The high-salt fractions demonstrate pronounced 67 kD ERα with low p-Ser-118 content and minimal evidence for 52 kD ERα variant expression. The solubilized pellet containing chromatin/nuclear matrix reveals both 67 kD and 52 kD ERα content as well as pronounced p-Ser-118 52 kD ERα formation.

FIG. 13, panels A through D, show that induction of NFκB DNA-binding in ER-positive breast cancer cells is prevented by inhibitors of NFκB activation. As described in the Examples, the level of NFκB DNA-binding activity was compared by EMSA using nuclear extracts prepared from untreated ER-positive cell lines MCF-7, MCF-7/HER2, and BT474 (panel A). Coincubation of probe and nuclear extracts with specific antibodies (p50 Ab, p65 Ab) produced supershifted bands (arrows) confirming the presence of p50 and p65 subunits within the DNA-bound NFκB complex from menadione treated (K3; 100 μM×30 min) MCF-7 nuclear extracts (panel B). Addition to the EMSA reaction of 50-fold excess cold competitor probe further demonstrated the specificity of NFκB complexes bound to the radiolabeled probe containing the κB consensus binding sequence (panels A-C). Menadione treated MCF-7 cells were also cultured in the presence of NFκB inhibitors MG132 (25 μM), PS341 (5 μM), PDTC (100 μM), or PA (50 μM) relative to vehicle treated controls (panels C and D). Cytosolic (C) or nuclear (N) extracts from treated MCF-7 cells were immunoblotted to demonstrate treatment induced changes in the cytosolic levels of IκBα inhibitor of NFκB and nuclear content of the p50 and p65 NFκB subunits, relative to β-actin loading controls (panel D).

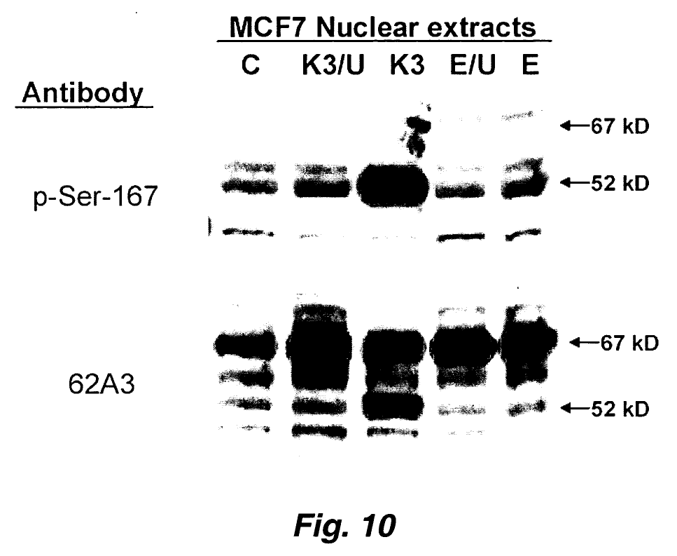

FIG. 14 shows that nhibition of NFκB activation by parthenolide (PA) sensitizes ERpositive/ErbB2-positive breast cancer cells to the antiestrogen tamoxifen. ERpositive/ErbB2-negative MCF-7 cells and ER-positive/ErbB2-positive MCF-7/HER2 and BT474 cells were treated with tamoxifen (TAM, 500 nM) alone, PA (5, 25, or 50 μM) alone, or the combination of TAM and PA for 24 hours. Cell viability was measured by sulforhodamine B (SRB) assay and scored as a percentage of control (vehicle treated) culture measurements. Results are expressed as the means±S.E.M. of triplicate wells; asterisks (*) indicate significant differences (p<0.001) for TAM+PA treatment conditions compared to PA treatments alone.

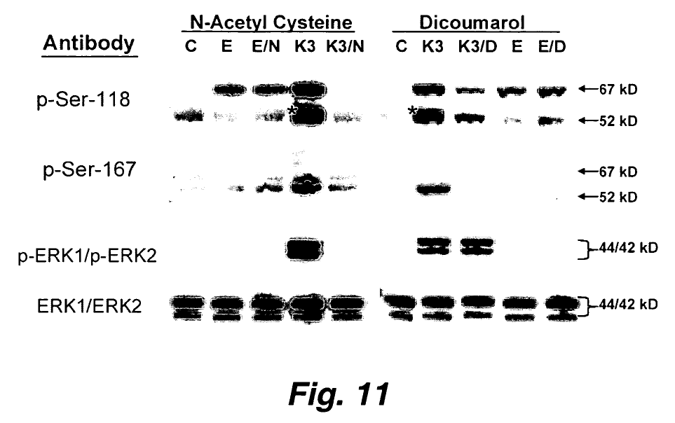

FIGS. 15A and 15B show that NFκB p50 and p65 subunit DNA-binding in ER-positive breast cancers is inversely related to the level of ER overexpression. Scatterplot showing significant correlation (rs=0.86; p<0.0001) between NFκB p50 and p65 subunit DNA-binding activities among the composite collective of 81 ER-positive breast cancer samples, with subunit DNA-binding activities quantified (arbitrary OD450 nm units) by independent ELISA based assays (FIG. 15A). Comparison of NFκB p50 and p65 subunit DNA-binding activities between tumor groups (FIG. 15B): Group A tumors (n=22), preselected for their higher level of ER overexpression (ER>100 fmol/mg); and Group B tumors (n=59), preselected for their lower level of ER overexpression (ER=21-87 fmol/mg, median=47 fmol/mg) and other known characteristics, as described in Methods. Asterisks (*) indicate significant differences in Group B NFκB subunit DNA-binding values from Group A values (p<0.0001).

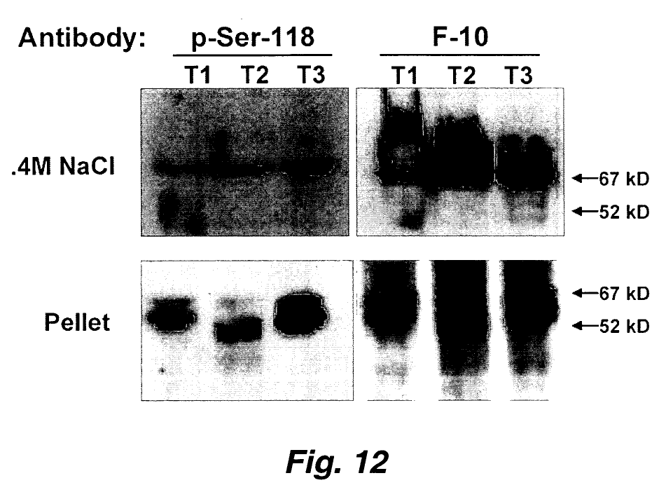

FIG. 16 shows that NFκB activation in ER-positive breast cancers correlates positively with ErbB2 protein expression. ErbB2 expression levels previously determined on the Group B tumor extracts are displayed in a scatter plot against the presently determined NFκB p50 and p65 DNA-binding values from each sample extract (as represented in the FIG. 15 bar graphs). Correlation coefficients (r_) and levels of significance (p values) for the p50 and p65 NFκB DNA-binding relationships to ErbB2 protein content (Units/mg) are shown. The dotted lines represent the clinically validated threshold level (500 U/mg) above which identifies ErbB2 overexpressing breast cancers (Eppenberger-Castori et al., 2001).

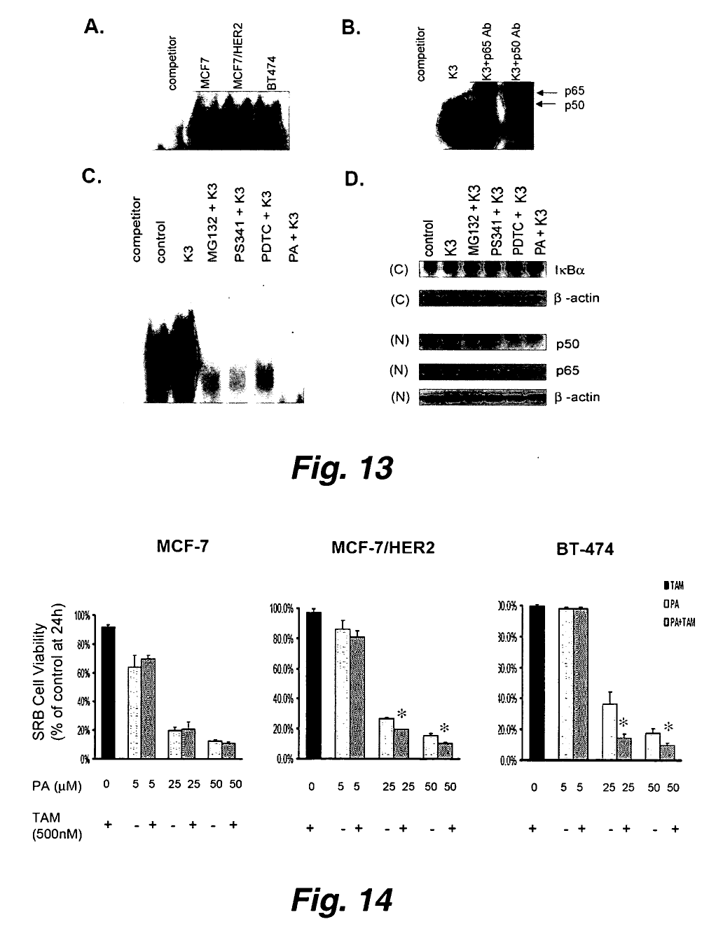

FIG. 17 shows that increased NFκB p50 DNA-binding activity correlates with increased AP-1 DNA-binding and increased uPA expression. Scatter plots and correlation coefficients (r) with levels of significance (p values) for the presently determined NFκB p50 DNAbinding values and the previously determined levels of uPA expression and AP-1 DNAbinding values for the Group B tumors.

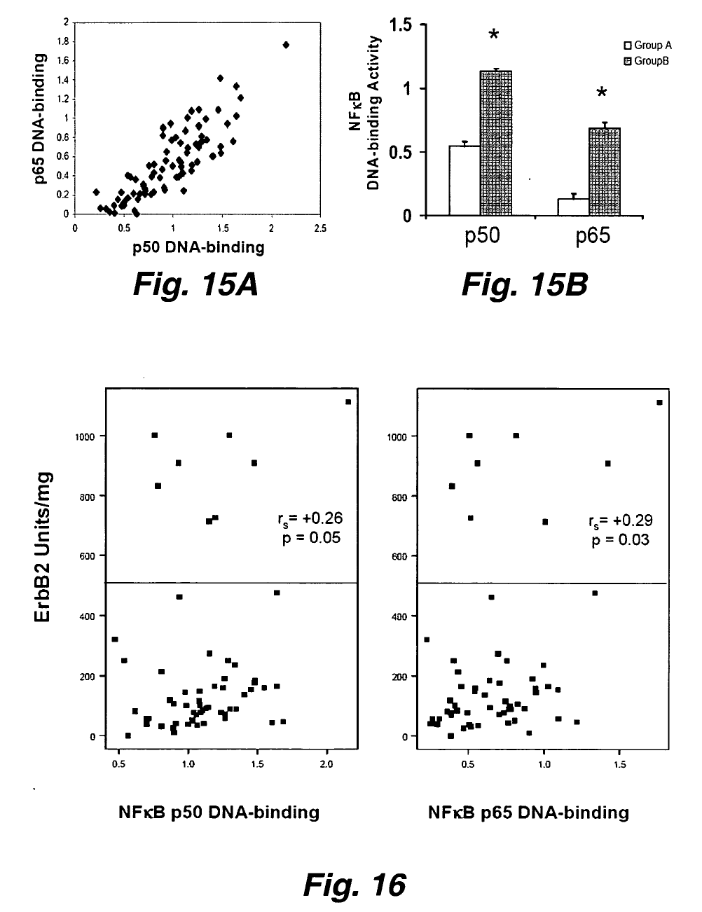

FIG. 18 shows that increased NF_B p50 DNA-binding in primary ER-positive breast cancers is associated with subsequent metastatic relapse. Patient clinical follow-up was recorded following excision and primary treatment of all the node-negative Group B cases, including subsequent development of all metastatic recurrences. Box plots show the increased NFκB p50 and p65 DNA-binding values from all the primary breast cancers that subsequently relapsed (13/59), as compared to the similarly staged ERpositive cases that did not relapse (46/59). By Mann-Whitney testing, the increase in median NFκB DNA-binding values did not reach statistical significance (p=0.08 for p50; p=0.18 for p65); however, in a univariate Cox regression model predicting disease-free survival, p65 DNA-binding was not significant (p=0.16), however p50 DNA-binding was significant (p=0.04).

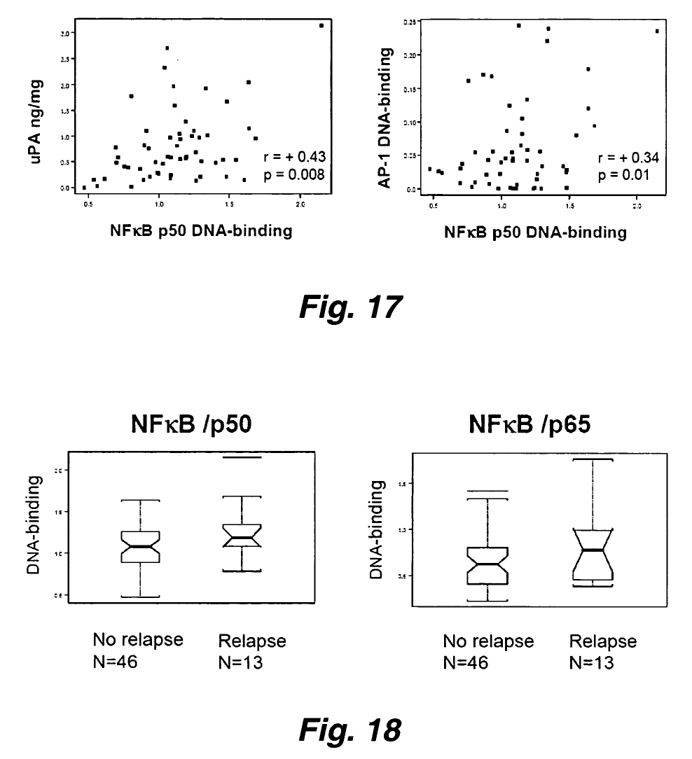

FIG. 19 shows that increased NFκB p50 DNA-binding, AP-1 DNA-binding, and uPA expression detected in primary ER-positive breast cancers are associated with reduced disease-free patient survival (DFS). In keeping with the association between increased NFκB DNA-binding and metastatic relapse as shown in FIG. 6, the above Kaplan-Meier curves indicate that higher vs. lower NFκB DNA-binding values are associated with different DFS outcomes; these DFS differences reach significance for p50 DNA-binding (p=0.04) but not for p65 DNA-binding (p=0.09). Consistent with the NFκB correlations shown in FIG. 17, higher vs. lower AP-1 DNA-binding and uPA expression values are also associated with significantly different DFS outcomes, as shown. The cutpoints dichotomizing Group B cases into higher vs. lower values of NFκB p50 DNA-binding (0.95), p65 DNA-binding (0.75), AP-1 DNA-binding (0.17), and uPA expression (1.8) were determined by regression tree analysis, as described in Methods. Values for uPA were not available on three of the cases, and AP-1 DNA-binding values were not available on five of the 59 Group B cases.

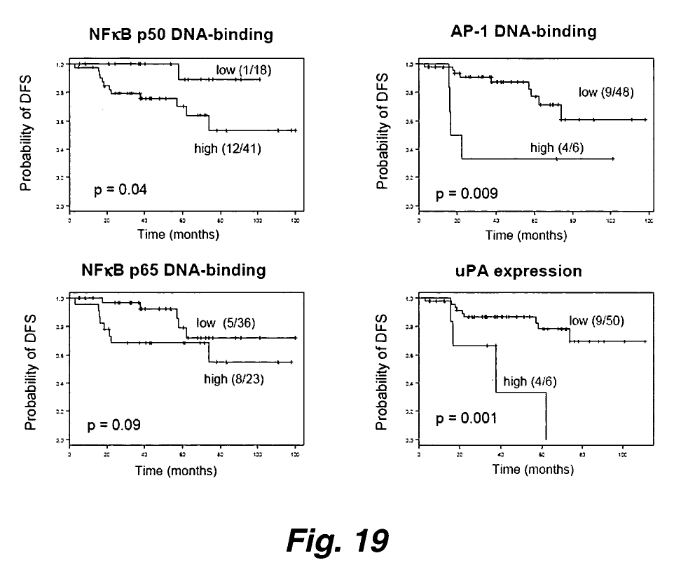

### DETAILED DESCRIPTION

This invention pertains to the identification of two novel epithelial signaling pathways in ER-positive breast cancer s and the discovery that the cellular biology and (likely also the clinical outcome) of ER-positive breast cancer cells is unexpectedly altered when these signaling pathways are activated.

The first pathway pertains to the discovery that NF-κB activation and/or DNA binding is implicated in the etiology of ER-positive breast (and other) cancers. Thus, ER-positive breast cancers with high NF-κB activation, which would previously only be considered for treatment by hormonal therapy are also targets for other kinds of therapy (e.g. NF-κB inhibitors) instead of or in addition to hormonal therapy. NF-κB thus also provides a prognostic/diagnostic marker for people less likely to respond to hormonal therapy.

In another embodiment, this invention pertains to the discovery that pathways involving ligand-independent quinine-mediated ER activation by posphorylation (e.g. on SER-118 and SER-167 residues of ER) and nuclear translocation of full-length (67 kDA) ER as well as the phorphorylating activation of a truncated and nuclear-localized ER variant (˜52 kDa), are implicated in the etiology of various cancers (e.g. ER-positive breast cancers). Inhibitors of MAPK and/or the vitamin K cycle can be used to inhibit ligand-independent activation of the ER and/or other steroid receptors. In addition, nuclear localization/phosphorylation of the ˜52 kDa ER isoforms provides a good prognostic to identify subjects undergoing ligand-independent receptor activation and thereby evaluate prognosis or alter treatment regimen. Moreover, the implication of quinines in the activation of steroid receptors has implications with respect to tumorigenesis and chemoprotection. Thus, chronic exposure to quinines or to quinine-inducing agents may have significant health effects.

I. NFκB in ER-Positive Epithelial Cancers.

The first of these pathways involves activation/DNA-binding by the well known oxidant- and stress-responsive survival and transcription activator complex NFκB (P40, P65 subunits), and important mediator of immune cell function and, more recently implicated in early-stage transformation and later stage progression of endocrine-independent and ER (alpha isoforms)-negative breast cancers (Nakshatri et al. (1997) Mol. Cell. Biol., 17: 3629-3639; Sovak et al. (1997) J. Clin. Investig., 100: 2952-2960; Biswas et al. (2000) Proc. Natl. Acad. Sci., USA, 97: 8542-8547; Romieu-Mourez et al. (2002) Cancer Res., 62: 6770-6778; Cao and Carin (2003) J. Mammary Gland Biol. & Neoplasia, 8: 215-223). Of note, these latter studies have specifically taught against the possible role of NFκB activation in the biology or clinical behavior of ER-positive breast cancer. In support of this, subcellular mechanisms have been identified showing that NFκB expression antagonizes ER expression.

Having recently shown that a subset of ER positive breast cancers exhibit molecular evidence of exposure to chronic oxidant stress, we looked for and found evidence that ER-positive breast cancer cells can increase NFκB DNA binding gin response to various oxidant stresses (e.g., H2020, redox-active quinines like menadione), and that a subset of untreated primary ER-positive breast cancers contain increased NFκB activation/DNA binding comparable to that found in ER-negative breast cancers. This increase in NFκB DNA-binding in some ER-positive breast cancers correlates with known oxidant stress-associated loss of SP1 DNA-binding and increase in AP1 DNA-binding, and associates with other indicators of poor clinical outcome and relative resistance to recurrence/met6astasis. This increase in NFκB DNA-binding (P50 and/or P65) subunits is readily measurable by either standard electrophoretic mobility shift assay (EMSA) or newer commercial ELISA-based DNA binding assays (e.g., TRANSAM kit by Active Motif).

Commercially available direct and indirect inhibitors of NFκB capable of downregulating NFκB activity/DNA binding exist that include, but are not limited to parthenolide, antioxidants like pyrrolidine dithiocarbamater (PDTC), proteasome inhibitors like PS-341/Bortezomib, and various other chemopreventive agents (Bharti et al. (2002) Biochem. Parmacol., 64: 883-888). Thus, use of such inhibitors and chemopreventive agents can now be anticipate dfor potential clinical use and targeted therapy against ER-positive/NFκB positive breast and other cancers and thus introduce a novel form of endocrine therapy that can be clinically effective alone or in conjunction with standard endocrine therapy (e.g., selective estrogen modulators, pure antiestrogens, or estrogen ablation in the form of aromatase inhibitors or medical/surgical oophorectomy).

II. NF-κB Inhibitors.

It was a surprising discovery that NF-κB activation and DNA binding appears to be a component involved in the etiology of ER-positive breast cancers (and presumably other cancers as well). Moveover, inhibition of NFκB expression, activation, and/or DNA binding is expected to be of use in the treatment of those ER-positive cancers characterized by elevated NF-κB activity.

NF-κB expression, activity, or DNA binding can be inhibited using any one or more of many inhibitors known to those of skill in the art. Such inhibitors include, but are not limited to various anti-oxidants (see, e.g., Table 1), proteasome and proteases inhibitors, e.g. that inhibit Rel/NF-kB (see, e.g., Table 2), phosphorylation and/or degredation inhibitors (see, e.g., Table 3), and various other inhibitors (see, e.g., Table 4). It is noted that the inhibitors identified herein are intended to be illustrative and not limiting.

TABLE 1
Anti-oxidants that have been shown to inhibit activation of NF-kB

a-lipoicacid
a-tocopherol
Agedgarlicextract
Anetholdithiolthione(ADT)
Applejuice
Astaxanthin
bis-eugenol
Butylatedhydroxyanisole(BHA)
Cepharanthine
CaffeicAcidPhenethylEster(3,4-dihydroxycinnamicacid, CAPE)
Carnosol
Carvedilol
CatecholDerivatives
Curcumin(Diferulolylmethane)
Dibenzylbutyrolactonelignans
Diethyldithiocarbamate(DDC)
Diferoxamine
DihydrolipoicAcid
Dilazep + fenofibricacid
Dimethyldithiocarbamates(DMDTC)
Curcumin(Diferulolylmethane)
Dimethylsulfoxide(DMSO)
Disulfiram
Ebselen
EPC-K1(phosphodiestercompoundofvitaminEandvitaminC)
Epigallocatechin-3-gallate(EGCG; greenteapolyphenols)
Ergothioneine
EthylPyruvate(Glutathionedepletion)
EthyleneGlycolTetraaceticAcid(EGTA)
Gamma-glutamylcysteinesynthetase(gamma-GCS)
Ganodermalucidumpolysaccharides
Ginkgobilobaextract
Glutathione
Hematein
IRFI042(VitaminE-likecompound)
Irontetrakis
L-cysteine
Lacidipine
Lazaroids
Magnolol
Manganesesuperoxidedismutase(Mn-SOD)
Melatonin
N-acetyl-L-cysteine(NAC)
Nacyselyn(NAL)
Nordihydroguaiariticacid(NDGA)
Orthophenanthroline
Phenolicantioxidants(Hydroquinoneandtert-butylhydroquinone)
Phenylarsineoxide(PAO, tyrosinephosphataseinhibitor)
Pyrrolinedithiocarbamate(PDTC)
Quercetin
Redwine
Ref-1(redoxfactor1)
Rg(3), aginsengderivative
Rotenone
S-allyl-cysteine(SAC, garliccompound)
Sauchinone
Tepoxaline(5-(4-chlorophenyl)-N-hydroxy-(4-methoxyphenyl)-N-methyl-
1H-pyrazole-3-propanamide)
VitaminC
VitaminEderivatives
a-torphrylsuccinate
a-torphrylacetate
PMC(2,2,5,7,8-pentamethyl-6-hydroxychromane)
YakuchinoneAandB

TABLE 2
Proteasome and proteases inhibitors that inhibit Rel/NF-kB

PeptideAldehydes:
ALLnL
(N-acetyl-leucinyl-leucynil-norleucynal, MG101)
LLM(N-acetyl-leucinyl-leucynil-methional)
Z-LLnV
(carbobenzoxyl-leucinyl-leucynil-norvalinal, MG115)
Z-LLL
(carbobenzoxyl-leucinyl-leucynil-leucynal, MG132)
Lactacystine, b-lactone
BoronicAcidPeptide
UbiquitinLigaseInhibitors
PS-341
CyclosporinA
FK506(Tacrolimus)
Deoxyspergualin
APNE(N-acetyl-DL-phenylalanine-b-naphthylester)
BTEE(N-benzoylL-tyrosine-ethylester)
DCIC(3,4-dichloroisocoumarin)
DFP(diisopropylfluorophosphate)
TPCK(N-a-tosyl-L-phenylalaninechloromethylketone)
TLCK(N-a-tosyl-L-lysinechloromethylketone)

TABLE 3
IκBα phosphorylation and/or degradation inhibitors

| Molecule | Inhibit IkBa's |
|---|---|
| Calagualine (fern derivative) | upstream of IKK (TRAF2-NIK) |
| LY29 and LY30 | PI3Kinase inhibitors |
| Pefabloc (serine protease inhibitor) | upstream of IKK |
| Rocaglamides (Aglaia derivatives) | upstream of IKK |
| Geldanamycin | IKK complex formation |
| BMS-345541 (4(2′-Aminoethyl)amino-1,8- | IKKa and IKKb kinase activity |
| dimethylimidazo(1,2-a) | |
| quinoxaline) | |
| 2-amino-3-cyano-4-aryl-6-(2-hydroxyphenyl) | IKKb activity |
| pryridine analog (compoud 26) | |
| Anandamide | IKKb activity |
| AS602868 | IKKb activity |
| BMS-345541 | IKK activity |
| Flavopiridol | IKK activity and RelA phosphor. |
| Jesterone dimer | IKKb activity |
| HB-EGF (Heparin-binding epidermal growth | IKK activity |
| factor-like growth factor | |
| LF15-0195 (analog of 15-deoxyspergualine) | IKK activity |
| Mild hypothermia | IKK activity |
| MX781 (retinoid antagonist) | IKK activity |
| Nitrosylcobalamin (vitamin B12 analog) | IKK activity |
| Survanta (Surfactant product) | IKK activity |
| PTEN (tumor suppressor) | Activation of IKK |
| Silibinin | IKKα activity |
| Sulfasalazine | IKKa and IKKb kinase activity |
| Piceatannol | IKK activity |
| Quercetin | IKK activity |
| Staurosporine | IKK activity |
| Wedelolactone | IKK activity |
| Betulinic acid | IKKa activity and p65 phosphorylation |
| Ursolic acid | IKKa activity and p65 phosphorylation |
| Thalidomide | IKK activity |
| Interleukin-10 | Reduced IKKa and IKKb expression |
| Anethole | Phosphorylation |
| Anti-thrombin III | Phosphorylation |
| Aspirin, sodium salicylate | Phosphorylation, IKKbeta |
| Azidothymidine (AZT) | Phosphorylation |
| BAY-117082 | Phosphorylation |
| (E3((4-methylphenyl)-sulfonyl)-2- | |
| propenenitrile) | |
| BAY-117083 | Phosphorylation |
| (E3((4-t-butylphenyl)-sulfonyl)-2- | |
| propenenitrile) | |
| Black raspberry extracts | Phosphorylation |
| Cacospongionolide B | Phosphorylation |
| Calagualine | Phosphorylation |
| Carbon monoxide | Phosphorylation |
| Chorionic gonadotropin | Phosphorylation |
| Cycloepoxydon; 1-hydroxy-2- | Phosphorylation |
| hydroxymethyl-3-pent-1-enylbenzene | |
| Extensively oxidized low density lipoprotein | Phosphorylation |
| (ox-LDL), 4-Hydroxynonenal (HNE) | |
| Gabexate mesilate | Phosphorylation |
| Glossogyne Tenuifolia | Phosphorylation |
| Hydroquinone | Phosphorylation |
| Ibuprofen | Phosphorylation |
| Indirubin-3′-oxime | Phosphorylation |
| Interferon-alpha | Phosphorylation |
| Methotrexate | Phosphorylation |
| Monochloramine | Phosphorylation |
| Nafamostat mesilate | Phosphorylation |
| Nitric Oxid (NO) | Phosphorylation |
| Oleandrin | Phosphorylation |
| Omega 3 fatty acids | Phosphorylation |
| Panduratin A (from Kaempferia pandurata, | Phosphorylation |
| Zingiberaceae) | |
| Petrosaspongiolide M | Phosphorylation |
| Prostaglandin A1 | Phosphorylation |
| Phytic acid (inositol hexakisphosphate) | Phosphorylation |
| Saline (low Na+ istonic) | Phosphorylation |
| Sanguinarine (pseudochelerythrine, 13- | Phosphorylation |
| methyl-[1,3]-benzodioxolo-[5,6-c]-1,3- | |
| dioxolo-4,5 phenanthridinium) | |
| Silymarin | Phosphorylation |
| SOCS1 | Phosphorylation |
| Statins (several) | Phosphorylation |
| Sulindac | IKK/Phosphorylation |
| THI 52 (1-naphthylethyl-6,7-dihydroxy- | Phosphorylation |
| 1,2,3,4- | |
| tetrahydroisoquinoline) | |
| Vesnarinone | Phosphorylation |
| YopJ (encoded by Yersinia | Phosphorylation |
| pseudotuberculosis) | |
| Acetaminophen | Degradation |
| a-melanocyte-stimulating hormone (a-MSH) | Degradation |
| Amentoflavone | Degradation |
| Aucubin | Degradation |
| beta-lapachone | Degradation |
| Capsaicin (8-methyl-N-vanillyl-6- | Degradation |
| nonenamide) | |
| Core Protein of Hepatitis C virus (HCV) | Degradation |
| Cyclolinteinone (sponge sesterterpene) | Degradation |
| Diamide (tyrosine phosphatase inhibitor) | Degradation |
| E-73 (cycloheximide analog) | Degradation |
| Ecabet sodium | Degradation |
| Electrical stimulation of vagus nerve | Degradation |
| Emodin (3-methyl-1,6,8- | Degradation |
| trihydroxyanthraquinone) | |
| Erbstatin (tyrosine kinase inhibitor) | Degradation |
| Estrogen (E2) | Degradation |
| Fosfomycin | Degradation |
| Fungal gliotoxin | Degradation |
| Gabexate mesilate | Degradation |
| Genistein (tyrosine kinase inhibitor) | Degradation; caspase cleavage of IkBa |
| Glimepiride | Degradation |
| Glucosamine sulfate | Degradation |
| gamma-glutamycysteine synthetase | Degradation |
| Hypochlorite | Degradation |
| IL-13 | Degradation |
| Intravenous immunoglobulin | Degradation |
| Isomallotochromanol and | Degradation |
| isomallotochromene | |
| Leflunomide metabolite (A77 1726) | Degradation |
| Losartin | Degradation |
| LY294002 (PI3-kinase inhibitor) [2-(4- | Degradation |
| morpholinyl)-8-phenylchromone] | |
| Murr1 gene product | Degradation |
| Neurofibromatosis-2 (NF-2) protein | Degradation |
| U0126 (MEK inhibitor) | Degradation |
| Pervanadate (tyrosine phosphatase inhibitor) | Degradation |
| Phenylarsine oxide (PAO, tyrosine | Degradation |
| phosphatase inhibitor) | |
| Pituitary adenylate cyclase-activating | Degradation |
| polypeptide (PACAP) | |
| Prostaglandin 15-deoxy-Delta(12,14)-PGJ(2) | Degradation |
| Resiniferatoxin | Degradation |
| Sesquiterpene lactones (parthenolide; | Degradation |
| ergolide; guaianolides) | |
| Thiopental | Degradation |
| Titanium | Degradation |
| TNP-470 (angiogenesis inhibitor) | Degradation |
| Stinging nettle (Urtica dioica) plant extracts | Degradation |
| Triglyceride-rich lipoproteins | Degradation |
| Vasoactive intestinal peptide | Degradation (and CBP-RelA interaction) |
| HIV-1 Vpu protein | TrCP ubiquitin ligase inhibitor |
| Epoxyquinone A monomer | IkBa ubiqutination inhibitor |
| Ro106-9920 (small molecule) | IkBa ubiqutination inhibitor |

TABLE 4
Miscellaneous inhibitors of NF-kB.

| Inhibitor Molecule | Detected Effect |
|---|---|
| Conophylline (Ervatamia microphylla) | Down regulated TNF-Receptors |
| MOL 294 (small molecule) | Redox regulated activation of NF- |
| | kB |
| Rhein | MEKK activation of NF-kB |
| apigenin (4′,5,7-trihydroxyflavone) | IkBa upregulation |
| beta-amyloid protein | IkBa upregulation |
| DQ 65-79 (aa 65-79 of the alpha helix of the alpha- | IkBa upregulation and IKK |
| chain of the class II HLA molecule DQA03011) | inhibition |
| C5a | IkBa upregulation |
| Glucocorticoids (dexamethasone, prednisone, | IkBa upregulation |
| methylprednisolone) | |
| IL-10 | IkBa upregulation |
| IL 13 | IkBa upregulation |
| IL-11 | IkBa, IkBb upregulation |
| Fox1j | IkBb upregulation |
| Dioxin | RelA nuclear transport |
| Astragaloside IV | Nuclear translocation |
| Atorvastatin | Nuclear translocation |
| Dehydroxymethylepoxyquinomicin (DHMEQ) | Nuclear translocation |
| 15-deoxyspergualin | Nuclear translocation |
| Disulfiram | Nuclear translocation |
| Estrogen enhanced transcript | Nuclear translocation |
| Gangliosides | Nuclear translocation |
| Glucorticoid-induced leucine zipper protein (GILZ) | Nuclear translocation |
| Heat shock protein 72 | Nuclear translocation |
| Leptomycin B (LMB) | Nuclear translocation |
| NLS Cell permeable peptides | Nuclear translocation |
| Nucling | RelA nuclear translocation |
| o,o′-bismyristoyl thiamine disulfide (BMT) | Nuclear translocation |
| Phalloidin | Nuclear translocation |
| Probiotics | RelA nuclear translocation |
| RelA peptides (P1 and P6) | Nuclear translocation |
| Retinoic acid receptor-related orphan receptor-alpha | Nuclear translocation |
| SC236 (a selective COX-2 inhibitor) | Nuclear translocation |
| Sphondin (furanocoumarin derivative from Heracleum | Nuclear translocation |
| laciniatum) | |
| ZUD protein | Activation of NF-kB; binds |
| | p105/RelA |
| ZAS3 protein | RelA nuclear translocation; DNA |
| | competition |
| Clarithromycin | nuclear expression |
| Triflusal | nuclear expression |
| HSCO (hepatoma protein) | Accelerates RelA nuclear export |
| 2-acetylaminofluorene | DNA binding |
| ADP ribosylation inhibitors (nicotinamide, 3- | DNA binding |
| aminobenzamide) | |
| 7-amino-4-methylcoumarin | DNA binding |
| Amrinone | DNA binding |
| Angiopoietin-1 | DNA binding |
| Artemisinin | DNA binding |
| Atrial Natriuretic Peptide (ANP) | DNA binding/IkBa upregulation |
| Atrovastat (HMG-CoA reductase inhibitor) | DNA binding |
| AvrA protein (Salmonella) | DNA binding |
| Baicalein (5,6,7-trihydroxyflavone) | DNA binding |
| Benfotiamine (thiamine derivative) | DNA binding |
| beta-catenin | DNA binding |
| beta-lapachone (a 1,2-naphthoquinone) | DNA binding |
| Biliverdin | DNA binding |
| Bisphenol A | DNA binding |
| Bovine serum albumin | DNA binding |
| Calcium/calmodulin-dependent kinase kinase | DNA binding |
| (CaMKK) (and increased intracellular calcium by | |
| ionomycin, UTP and thapsigargin) | |
| Calcitriol (1a,25-dihydroxyvitamine D3) | DNA binding |
| Caprofin | DNA binding |
| Capsiate | DNA binding |
| Catalposide (stem bark) | DNA binding |
| Cat's claw bark (Uncaria tomentosa; Rubiaceae) | DNA binding |
| Chitosan | DNA binding |
| Clarithromycin | DNA binding |
| Commerical peritoneal dialysis solution | DNA binding |
| Cytochalasin D | DNA binding |
| (kB site) Decoy oligonucleotides | DNA binding |
| Diamide | DNA binding |
| Diarylheptanoid 7-(4′-hydroxy-3′-methoxyphenyl)-1- | DNA binding |
| phenylhept-4-en-3-one | |
| DTD (4,10-dichloropyrido[5,6:4,5]thieno[3,2-d′:3,2- | DNA binding |
| d]-1,2,3-ditriazine) | |
| E3330 (quinone derivative) | DNA binding |
| ent-kaurane diterpenoids (Croton tonkinensis leaves) | DNA binding |
| Epoxyquinol A (fungal metabolite) | DNA binding |
| Erythromycin | DNA binding |
| Fibrates | DNA binding |
| Flunixin meglumine | DNA binding |
| Flurbiprofen | DNA binding |
| Ganoderma lucidum (fungal dried spores or fruting | DNA binding |
| body) | |
| Glycyrrhizin | DNA binding |
| Hematein (plant compound) | DNA binding |
| Herbal compound 861 | DNA binding |
| Herbimycin A | DNA binding |
| Hydroxyethyl starch | DNA binding |
| Hypericin | DNA binding |
| Herperosmolarity | DNA binding |
| Hypoethyl starch | DNA binding |
| Hypothermia | DNA binding |
| Hydroquinone (HQ) | DNA binding |
| Interleukin 4 (IL-4) | DNA binding |
| IkB-like proteins (encoded by ASFV) | DNA binding |
| Kamebakaurin | DNA binding |
| Kaposi's sarcoma-associated herpesvirus K1 protein | DNA binding |
| Ketamine | DNA binding |
| KT-90 (morphine synthetic derivative) | DNA binding |
| Lovastatin | DNA binding |
| Macrolide antibiotics | DNA binding |
| 2-methoxyestradiol | DNA binding |
| Metals (chromium, cadmium, gold, lead, mercury, zinc, | DNA binding |
| arsenic) | |
| Mevinolin, 5′-methylthioadenosine (MTA) | DNA binding |
| Monomethylfumarate | DNA binding |
| Myxoma Virus MNF | DNA binding |
| NDPP1 (CARD protein) | DNA binding |
| N-ethyl-maleimide (NEM) | DNA binding |
| Nicotine | DNA binding |
| Extracts of Ochna macrocalyx bark | DNA binding |
| Leucine-rich effector proteins of Salmonella & Shigella | DNA binding |
| (SspH1 and IpaH9.8) | |
| Omega-3 fatty acids | DNA binding |
| p8 | DNA binding |
| 1,2,3,4,6-penta-O-galloyl-beta-D-glucose | DNA binding |
| p202a (nterferon inducible protein) | DNA binding by p65 and p50/p65; |
| | increases p50 |
| PC-SPES (8 herb mixture) | DNA binding |
| Pentoxifylline (1-(5′-oxohexyl) 3,7-dimetylxanthine, | DNA binding |
| PTX) | |
| 6(5H)-phenanthridinone and benzamide | DNA binding |
| Phenyl-N-tert-butylnitrone (PBN) | DNA binding |
| Phyllanthus amarus extracts | DNA binding |
| Pioglitazone (PPARgamma ligand) | DNA binding |
| Pirfenidone | DNA binding |
| Prostaglandin E2 | DNA binding |
| Protein-bound polysaccharide (PSK) | DNA binding |
| Pyrithione | DNA binding |
| Quinadril (ACE inhibitor) | DNA binding |
| Raxofelast | DNA binding |
| Rebamipide | DNA binding |
| Ribavirin | DNA binding |
| Rifamides | DNA binding |
| Rolipram | DNA binding |
| Sanggenon C | DNA binding |
| Secretory leukocyte protease inhibitor (SLPI) | DNA binding |
| Serotonin derivative (N-(p-coumaroyl) serotonin, SC) | DNA binding |
| Siah2 | DNA binding |
| Silibinin | DNA binding |
| Sulfasalazine | DNA binding |
| SUN C8079 | DNA binding |
| Surfactant protein A | DNA binding |
| T-614 (a methanesulfoanilide anti-arthritis inhibitor) | DNA binding |
| Tanacetum larvatum extract | DNA binding |
| Taurine + niacine | DNA binding |
| Tyrphostin AG-126 | DNA binding |
| Vascular endothelial growth factor (VEGF) | DNA binding |
| Wogonin (5,7-dihydroxy-8-methoxyflavone) | DNA binding |
| APC0576 | Transactivation |
| Blueberry and berry mix (Optiberry) | Transactivation |
| Chromene derivatives | Transactivation |
| D609 (phosphatidylcholine-phospholipase C inhibitor) | Transactivation |
| Ethyl 2-[(3-methyl-2,5-dioxo(3-pyrrolinyl)) | Transactivation |
| amino]-4-(trifluoromethyl) | |
| pyrimidine-5-carboxylate | |
| Cycloprodigiosin hycrochloride | Transactivation |
| Dimethylfumarate (DMF) | Transactivation |
| Fructus Benincasae Recens extract | Transactivation |
| Glucocorticoids (dexametasone, prednisone, | Transactivation |
| methylprednisolone) | |
| Phenethylisothiocyanate | Transactivation |
| Pranlukast | Transactivation |
| Psychosine | Transactivation |
| Quinazolines | Transactivation |
| Resveratrol | RelA nuclear localization and |
| | transactivation |
| RO31-8220 (PKC inhibitor) | Transactivation |
| Saucerneol D and saucerneol E | Transactivation |
| SB203580 (p38 MAPK inhibitor) | Transactivation |
| Tranilast [N-(3,4-dimethoxycinnamoyl)anthranilic | Transactivation |
| acid] | |
| 3,4,5-trimethoxy-4′-fluorochalcone | Transactivation |
| Triptolide (PG490, extract of Chinese herb) | Transactivation |
| Uncaria tomentosum plant extract | Transactivation |
| LY294,002 | Transactivation |
| Mesalamine | RelA phosphorylation & |
| | transactivation |
| PTX-B (pertussis toxin binding protein) | RelA phosphorylation and |
| | transactivation |
| Adenosine | Transactivation |
| 17-allylamino-17-demethoxygeldanamycin | Transactivation |
| 6-aminoquinazoline derivatives | Transactivation |
| Luteolin | p65 Transactivation |
| Manassantins A and B | p65 Transactivation |
| Qingkailing and Shuanghuanglian (Chinese medicinal | Transactivation |
| preparations) | |
| Tetrathiomolybdate | Transactivation |
| Trilinolein | Transactivation |
| Troglitazone | Transactivation |
| Wortmannin (fungal metabolite) | Transactivation |
| Rifampicin | Glucocorticoid receptor modulation |

In certain embodiments, the NKkB inhibitors include, but are not limited to sesquiterpene lactones such as parthenolide and/or parthenolide-like compounds (e.g. parthenolide analogues) (see, e.g., U.S. Pat. No. 5,905,089).

It is also noted that, as shown in the examples, it was a discovery of this invention that inhibiting NFkB can sensitize some ER-positive breast cancers to hormonal therapeutics including antiestrogens. Thus, in certain embodiments, this invention contemplates the use of NFkB inhibitors sequentially or in combination with hormonal therapeutics (e.g., various antiestrogens). Without being bound to a particular theory it is believe that the NFkB inhibitors will be particular effective when used with endocrine agents, especially against high-risk ER-positive breast cancers with elevated NFkB activity.

Thus, in certain embodiments, one or more NFkB inhibitors (e.g., parthenolide, parthenolide analogues, etc.) are used sequentially (before or after) or in combination with one or more hormonal therapeutics (e.g. antiestroges such as tamoxifen, 2-(4-Hydroxy-phenyl)-3-methyl-1-[4-(2-piperidin-1-yl-ethoxy)-benzyl]-1H-indol-5-ol hydrochloride (ERA-923), and the like.

III. Ligand-Independent Quinine-Mediated ER Activation.

It was also a surprising discovery that pathways involving ligand-independent quinine-mediated ER (alpha isoforms) activation by posphorylation (e.g. on SER-118 and SER-167 residues of ER) and nuclear translocation of full-length (67 kDA) ER as well as the phorphorylating activation of a truncated and nuclear-localized ER variant (˜52 kDa), characterized by antibody mapping as a previously reported delta 7 ER splice variant are implicated in the etiology of various cancers (e.g. ER-positive breast cancers).

Without being bound to a particular theory, it is believed that the nuclear localization of this ER variant has not be previously established. We have altered the standard tumor/cell extraction process for analysis of total ER to include a nuclear a nuclear chromatin solubilization step, and have now definitely shown that much of phosphorylated 67 kDa ER and virtually all of the phosphorylated ˜52 kDA ER variant is present in this nuclear chromatin fraction and has thus been undetectable and/or ignored in previous analyses of ER and ER phorphorylation.

We show that standard estrogenic ligand (e.g., estradiol) activation of ER expressed in breast cancer cells like MCF-7 or T47D results in a low level of and a different profile of ER phosphorylation as that produced by growth factor ligands (e.g. EGF, neuregulins) or tumor promoters/cell signaling activators like phorbol esters (TPA, PMA). Uniquely different from known, these ER activators are the profoundly potent effects of the redox-actigve quinine known as menadione (vitamin K3) and other more weakly reactive quinines including those produce from estrogen catechol metabolism.

In particular, and unlike the estrogenic ligands, receptor binding growth factors, or PKC/PKA-activating tumor promoters like TPM/PMA, vitamine K strongly phosphorylates both Ser-18 and Ser-167 in both 67 kDa and ˜52 kDA ER, translocating and fixing these ER species within the chromatin faction where they are unextractable by the typical ER and cell/tumor lysate procedures. This likely accounts for recently observed discrepancies between cell lysate ER assays and immunohistochemical phosphor-Ser118 ER results on breast cancer cell lines and primary tumors (Lannigan (2003) Steroids, 68: 1-9; Murphy et al. (2004) Clin. Cancer. Res., 10: 1354-1359).

As for the other known ligand-dependant and ligand-independent pathways to ER phosphorylation, quinine activation and posphorylation of ER is associated with MAPK activation and can be prevented by MAPK inhibitors (e.g. UO126). Unlike other known ligand-dependent and ligand-independent ER activating pathways, quinine activation and phosphorylation of ER can be prevented by inhibitors of the vitamin K cycle (including, but not limited to dicoumarol and warfarin-related compounds) which do not effect quinine activation of MAPK.

This dependence of ER activation by vitamin K-like quinines on the vitamin K cycle, and its ability to be inhibited/prevented by dicoumarol specifically indicates involvement by one or more members of the NAD(p)H quinine reductase (NQQR) family as a hitherto unrecognized path toward ligand-independent ER phorphorylation and activation. Thus, NQOR and other pathway components in the vitamin K cycle (including gamma-carboxyglutate carboxylase and vitamin K epoxide reductase) are believed to be of value for their prognostic and/or predictive utility in conjunction with ER and ER variant levels, phorphorylation of ER and ER variants, and in relation to MAPK activation. Likewise, excess exposure or endogenous metabolism generating ER phosphorylating and activating quinines like menadione/vitamin K3 may be seen as predisposing risk factors for development and/or progression of ER-positive breast cancer. Also, previously unrecognized connections to the vitamin K cycle may now be seen as important risk factors for developing ER-positive breast cancers (or other cancers characterized by activation of steroid hormone receptors). For example, the tight correlation between ER overexpression in breast cancers and activation of the AXL growth factor receptor may be critically dependent on excess activity by vitamin K-dependent gamma-carboxyglutamate carboxylase in generating excess local levels of the AXL ligand, GAS6. Likewise, other similarly modified membrane proteins dependent on the vitamin K cycle may be intimately linked to ligand-independent ER activation and the development of ER-positive breast cancers.

Novel therapeutics targeting this newly discovered ER cross-talking pathway, and even clinical use of vitamin K analogs like dicoumarol, may be anticipated as therapeutic and/or prevention approaches against ER-positive breast cancers or its preneoplastic precursors (e.g. DCIS, atypical hyperplasia, etc.).

IV. MAPK and Quinone Inhibitors.

It was a surprising discovery that ligand-independent activation of steroid hormone receptors can be mediated by quinines and that inhibitors of MAPK and/or the vitamin K pathway can be used to inhibit such ligand-independent activation. MAPK inhibitors and vitamin K pathway inhibitors (e.g. K3 inhibitors) are known to those of skill in the art. Thus, for example, MAPK inhibitors include, but are not limited to UO126, CNI-1493, SB-242235, PD 98059, ALX-385-008 (Apigenin), ALX-270-328 (2-(4-Chlorophenyl)-4-(fluorophenyl)-5-pyridin-4-yl-1,2-dihydropyrazol-3-one), ALX-350-290 (Debromohymenialdisine), ALX-350-289 (10Z-Hymenialdisine), LKT-H9861 (Hypericin), ALX-350-030 (Hypericin native), ALX-350-258 (Parthenolide), ALX-385-023 (PD 98,059), ALX-270-258 (PD 169,316), ALX-270-324 (Raf1 Kinase Inhibitor I), ALX-270-259 (SB202190), ALX-270-268 (SB202190 hydrochloride), ALX-270-179 (SB203580), ALX-270-325 (SB220025), ALX-270-351 (SB239063), ALX-270-257 (SC68376), ALX-270-260 (SKF-86002), ALX-270-237 (U0126), ALX-270-336 (ZM 336372), and the like.

Similarly, inhibitors of the vitamin K cycle are also known to those of skill in the art. Thus, for example warfarin and warfarin analogues are inhibitors of vitamin K-dependent gamma-carboxylation. A variety of other anti-coagulants also act as vitamin K inhibitors.

### EXAMPLES

The following examples are offered to illustrate, but not to limit the claimed invention.

### Example 1

### Ligand-Independent, MAPK-Dependent Activation and Serine Phosphorylation of Wild-Type (67 kDa) and Delta7 (˜52 kDa) Isoform of Estrogen Receptor Alpha by the Redox Active Quinone, Vitamin K3/Menadione

Ligand-independent activation of estrogen receptor alpha (ER) by membrane receptor-induced kinase signaling and phosphorylation of key serine residues(e.g., Ser-118) in the trans activating domain of ER is well described. Studying the varied effects of oxidants on ER structure and function, we treated ER-positive breast cancer MCF-7 and T47D cells growing in charcoal-stripped media with/without either thiol-reversible oxidants (e.g., H202, diamide) or the thiol-irreversible redox active and arylating quinone, vitamin K3/menadione (1,2-naphthooquinone; 100 μM×30 min). Both types of oxidants induced a dose-dependent loss of ER DNA-binding. Menadione, but not the thiol-reversible oxidants, induced Ser-118 phosphorylation and nuclear translocation of 67 kDa (wild-type) ER. Additionally, this quinone induced Ser-118 phosphorylation and nuclear translocation of an abundant ˜52 kDa ER isoforms with epitope characteristics of the expressed delta7 ER mRNA splice variant reported to act as a suppressor of wild-type ER. Treatment with 10 nM estradiol (E2) also produced Ser-118 phosphorylation and nuclear translocation of 67 kDa ER, but did not similarly affect the ˜52 kDa ER isoform. The quinone-induced phosphorylation of Ser-118 in 67 kDa ER and ˜52 kDa ER isoform could be completely blocked by the MAPK inhibitor UO126, which had minimal effect on Ser-118 phosphorylation induced by E2. Inhibitors of the PI3/AKT and p38 kinase pathways had little impact on menadione-induced Ser-118 phosphorylation. Interestingly, treatment with either an arylating and non-redox active quinone (p-benzoquinone) or a redox cycling and non-arylating quinone (DMNQ) produced no Ser˜118 phosphorylation of ER when administered at equitoxic doses and compared to menadione. Longer menadione exposures (4-6 hr) further enhanced activation of the ˜52 kDa delta7 ER isoform while diminishing that of 67 kDa ER. Immunoblots of ER-positive breast tumor lysates have identified numerous cases in which Ser-118 phosphorylation appears greatest on ER isoforms with structural and immunologic features comparable to the ER delta7 splice variant, despite the pronounced abundance of less phosphorylated 67 kDa ER. These findings suggest that the ˜52 kDa delta7 ER isoform is a primary target for ligand-independent activation by redox active and arylating quinones like menaqinone.

### Example 2

### Increased NF-κB Activation Identifes an Oxidatively-Stressed and Clinically More Aggressive Subset of Estrogen Receptor (ER)-Positive Breast Cancers

Recent studies indicate that NFKB is required for mammary gland development and may be involved in the etiology of breast cancer. In particular, progression from hormone-sensitive estrogen receptor (ER)-positive to hormone-resistant ER-negative breast cancer has ‘been shown to be associated with marked upregulation and activation of NF-κB, as measured by nuclear translocation and/or DNA-binding by NFκB subunits (e.g., p50, p65), and consistent with the fact that ER and NF-κB appear to mutually inhibit the transcriptional activity of one another. Since hormone-resistant subsets of ER-positive breast cancer have long been clinically recognized but are difficult to predict at presentation, we have explored the hypothesis that such ER-positive subsets, potentially induced by increased exposure to oxidative stress, can be identified by increased NF-κB activation.

Oxidative stress in the form of the redox active quinone, menadione/vitamin K3 applied to MCF-7 breast cancer cultures (10 μM, 30 min) increases >3-fold nuclear translocation of both NF-κB p50 and p65 subunits as compared to control treated cells.

Consistent with the proposed NFKB down-regulating anti-tumor mechanism of proteasome inhibitors, treatment of these ER-positive cells with MG-132 (10 μM, 30 min) produced a 60% decrease in the DNA-binding of both p50 and p65 subunits (TransAM assay”, ActiveMotif). We compared NF-κB subunit DNA-binding in two groups of ER-positive breast cancers, one with higher ER content (≧100 fmol/mg; mean 380 fmol/mg ER) and one with lower ER content (20-99 fmol/mg); the latter group had previously been reported showing age-associated oxidative stress markers (loss of Sp1 DNA-binding, increased phospho-Erk5, lower PR). The group with lower ER content (n=31) showed a statistically significant (p<0.001) two-fold higher mean level of NF-κB subunit DNA-binding as compared to the group with higher ER content (n=22). Among the lower ER content breast tumors, there appeared a negative correlation trend (r=−0.2) between NF-κB/p50 DNA-binding and level of SpI DNA-binding, and a significant positive correlation (r=0.4, p<0.05) between NF-κB/p65 DNA-binding and level of AP-1 DNA-binding. Since increased NF-κB DNA-binding is known be associated with increased AP-1 DNA-binding in ER-negative vs. ER-positive breast cancer cell lines, and we have previously shown that AP-I DNA-binding significantly increases in ER-positive breast cancers that become hormone-resistant, we conclude that oxidative stress-induced NF-κB activation may be involved in the mechanism.

### Example 3

### Quinone-Induced and Ligand-Independent Phosphorylation of Estrogen Receptor Alpha (ERα) and a Breast Cancer Expressed Nuclear ERα Variant

In this example, we used ERα positive human breast cancer cell lines (MCF7, T47D) as model systems to study the response of endogenously expressed ERα protein to cell treatment with a redox-stressing quinone, the vitamin K analog menadione (K3). K3 is a widely used quinone capable of reversible redox-cycling (generating reactive oxygen species, ROS) and irreversible Michael addition-type arylation of various intracellular proteins with available cysteine (Cys) residues (25, 26), including ERα (27). We observed that K3 treatment of these cells in culture induces rapid ligand-independent activation and Ser-118 phosphorylation of endogenous 67 kD ERα. Analysis of this response further revealed that K3 induced Ser-118 and Ser-167 phosphorylation of a 52 kD ERα variant associating with the chromatin/nuclear matrix of these cells. This 52 kD ERα nuclear variant is also found expressed and ser-118 phosphorylated in some ERα-positive breast cancer samples.

Breast cancer cells (MCF7, T47D) treated with the redox-stressing quinone, menadione (K3; >50 μM, >30 min), showed rapid nuclear translocation and serine (Ser)-118 phosphorylation of 67 kD ERα as well as ser-118 and ser-167 phosphorylation of a 52 kD nuclearly located ERα variant. Epitope mapping with a panel of ERα antibodies demonstrated that the 52 kD variant possesses amino (N)-terminal, exon 4 and carboxy (C)-terminal epitopes. Sequencing of selected RT-PCR products revealed cell expression of two in-frame ERα splice variants (exon 5,6 or exon 6,7 deletions) with size and epitope features consistent with the 52 kD nuclear variant. The 52 kD variant associated with the bound chromatin/nuclear matrix fraction; and ERα-positive breast cancers also contain detectable levels of this nuclear-bound and ser-118 phosphorylated variant. Inhibition of MAPK (U0126), but not PI-3 kinase (LY294002), JNK (SP600125), or p38 kinase (SB203580) pathways, suppressed K3 activation of 67 kD and 52 kD ERα variant. Dicoumarol, an inhibitor of NADPH quinone oxidoreductase-1 (NQO1), had no affect on MAPK activation but suppressed K3 induced phosphorylation of 67 kD and 52 kD ERα. These findings point to a novel ligand-independent and NQO 1-dependent mechanism by which a redox stressing quinone differentially activates wildtype and variant forms of ERα.

Results

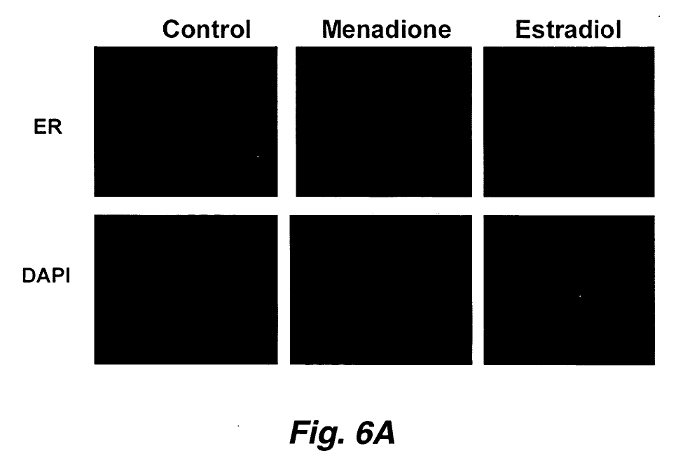

To examine quinone-induced redox stress on unliganded ERα, MCF7 and T47D breast cancer cells were grown and treated in phenol-free medium supplemented with 10% charcoal stripped serum As shown in FIG. 6A, by eliminating ligand exposure the endogenously expressed MCF-7 ERα is found primarily sequestered in the cytoplasm. After 30 min of cell exposure to K3 (100 μM) the majority of this cytoplasmic ERα has translocated to the nucleus, comparable to the nuclear translocation induced by a 30 min treatment with estradiol (E2, 10 nM).

The cytoplasmic to nuclear translocation of ERα induced by K3 prompted immunoblot analyses of these two cell compartments to detect receptor species reactive to a panel of ERα antibodies. The Westerns shown in FIG. 6B of cytoplasmic and nuclear extracts from MCF7 cells treated as in FIG. 6A were probed with antibodies specific for epitopes within the N-(62J3) and C-(F-10) terminal regions of ERα; they show that the cytoplasmic fractions contain only wildtype 67 kD ERα but the nuclear fractions contained 67 kD ERα and a 52 kD ERα species as detected by both antibodies.

While the N-terminal specific ERα antibody also detected a ˜60 kD immunoreactive species in the nuclear fractions, this additional ERα variant was not further pursued. Consistent with the immunoflouresence results shown in FIG. 6A, the immunoblot detected cytoplasmic 67 kD ERα that appeared more abundant in the control extracts relative to K3 and E2 treated extracts, while the opposite expression profiles were observed in the nuclear extract immunoblots. FIG. 6C shows the Cterminal epitope reactivity of the 52 kD ERα nuclear variant by comparing the exon-8 specific D75 monoclonal antibody (epitope between aa 554-570, Geoffrey Greene personal communication) with the primarily exon-7 specific H222 monoclonal antibody (epitope between aa 467-528 with exon-7 ending at aa 517, Geoffrey Greene personal communication). While the D75 antibody detects the 52 kD ERα variant relative to 67 kD ERα in similar proportions as detected by the C-terminal antibody F-10, the H222 antibody shows only very weak immunoreactivity against the 52 kD ERα variant, suggesting loss of exon-7 specific epitopes. Across a panel of seven different antibodies capable of detecting 67 kD ERα by Western analyses, only H222 failed to show good immunoreactivity with the 52 kD ERα variant as expressed in MCF7 and T47D cells.

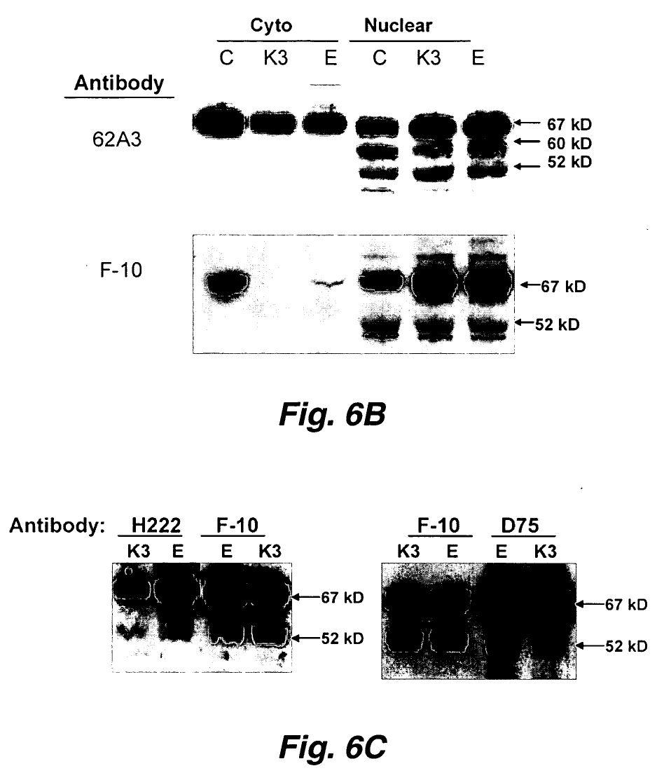

To investigate the nuclear sequestration of the 52 kD ERα variant, MCF7 nuclei were isolated following control, K3 or E2 treatment of cell cultures (as described in FIG. 6), and the nuclei were then partitioned into a high-salt (0.42 M NaCl) extractable fraction and a residual nuclear pellet fraction consisting primarily of chromatin/nuclear matrix and solubilized by DNase-1 digestion and SDS detergent treatment. Western blots of the high-salt extracted nuclear fractions and the solubilized chromatin/nuclear matrix fractions were probed with the ERα exon-4 specific antibody, SRA-1000, and the C-terminal specific antibody, D75, as shown in FIG. 7. Under all treatment conditions, the 52 kD ERα nuclear variant was found tightly associated within the chromatin/nuclear matrix fraction. Surprisingly, following K3 cell treatment the 67 kD ERα was also found primarily within this chromatin/nuclear matrix fraction, in contrast to E2 cell treatment which resulted in the majority of nuclear translocated 67 kD ERα to be extractable within in the high-salt nuclear fraction. To confirm the efficiency of the nuclear extractions as shown in FIG. 7, probing with an antibody for 44/42 kD ERK1/2 showed that high-salt extraction removed virtually all of the nuclear ERK1/2 from control, E2 and K3 treated nuclei.

Containing epitopes that map to the N-terminal, exon-4 and C-terminal regions of ERα, the 52 kD ERα nuclear variant appeared to represent a previously uncharacterized receptor variant. The corresponding mRNA transcript would be required to retain the wildtype reading frame from exons 1, 2, and 4 as well as that of exon 8, which encodes the C-terminal region of ERα. Previous RTPCR studies of various cell lines, normal tissues and ERα-positive tumors have identified numerous ERα variants usually with single exon deletions, most commonly missing exons 3, 4, 5 or 7, but none predicting a 52 kD protein with the epitope features shown in the immunoblots of FIGS. 6 and 7. Inspection of the ERα exon/intron genomic structure (available at the Ensamble database, http://www.ensembl.org/Homo_sapiens/exonview?transcript=ENST00000206249&db=core) shows that exons 5, 6 and 7 introduce frame-shifts of +1, +2 and +1 respectively, and introduce contributions of approximately 5.0 kD, 5.0 kD and 6.7 kD respectively to the molecular weight of ERα, suggesting that simultaneous deletion of exons 5 and 6 or exons 6 and 7 would to produce a splice variant capable of encoding a ˜52 kD ERα with preserved in-frame expression of exon-8.

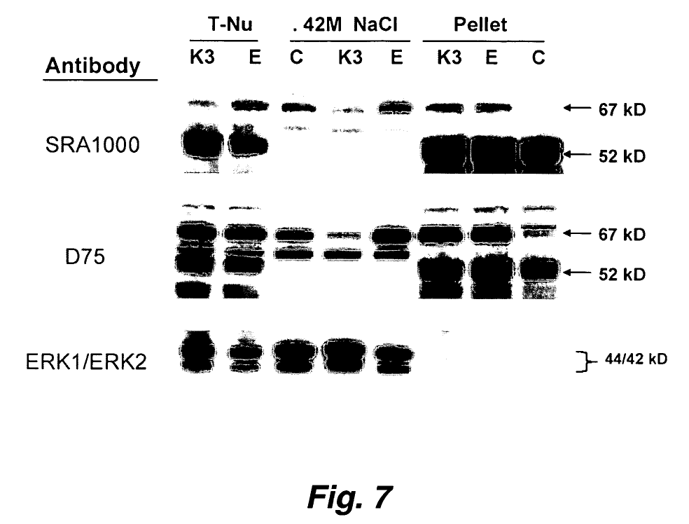

To search for such dual deletion splice variants, total RNA from MCF7 cells growing in either normal or charcoal stripped serum was primed with oligo dT, reverse transcribed (RT) and aliquots of the RT reaction were analyzed by PCR using either a previously described exon4/exon-8 primer pair or an exon-4/exon-7 primer pair (17). As shown in FIG. 8A, gel electrophoresis of the PCR products produced by the exon4/exon-8 primer pair revealed prominent bands at 600 bp and 460 bp in addition to the wildtype ERα band at 760 bp. Also shown in FIG. 8A are aliquots of the gel-purified and reamplified 600 bp and 460 bp bands used for sequencing. Consistent with its size and previously published description, sequencing of the 600 bp band revealed an exon-7 deleted variant (14-19). However, the 460 bp band, which had previously been described as a double deletion missing exons 5 and 7 (17 ), was found to also contain a similarly sized exon-6 and exon-7 double deletion. As shown in FIG. 8B, digestion of the 460 bp band with BglII, which cuts once in exon-6, eliminated most of the 460 bp band and produced a diagnostic doublet with fragments of 246 bp and 211 bp in length (FIG. 8B, lane 1), consistent with an exon-5 and exon-7 double deleted product that retains exon-6. Digestion of this 460 bp band with NcoI, which cuts once in exon-5, eliminated only ˜5% of the 460 bp band and produced fragments of length 302 bp and 160 bp (FIG. 8B, lane 2), consistent with an exon-6 and exon-7 double deleted product that retains exon-5. Sequencing of the gel-purified 460 bp band which remained following NcoI digestion confirmed it to be an exon-5 and exon-7 deleted variant while sequencing the gel-purified 460 bp band which remained following BglII digestion confirmed it to be a precise exon-6 and exon-7 deleted ERα variant as shown in FIG. 8D.

Gel electrophoresis of the PCR products produced by the exon4/exon-7 primer pair produced in addition to the 568 bp wildtype band, a band at 424 bp and a band of secondary intensity at 295 bp (FIG. 8C, lane 1). Sequencing of the 424 bp band revealed the previously described exon-5 deleted variant while sequencing of the 295 bp band revealed a precise exon-5 and exon-6 deleted variant (FIG. 8D). Thus, while the abundance of ERα variant transcripts harboring simultaneous deletions of exons-5 and exon-6 or exon-6 and exon-7 are clearly less than that of the full-length transcripts or variants with either single exon deletions or the exon-5 and exon-7 double deletion, the two newly described double deletion variants missing exon-6 are the only detectable transcripts capable of encoding a 52 kD ERα variant receptor with the epitope features shown in FIGS. 6 and 7.

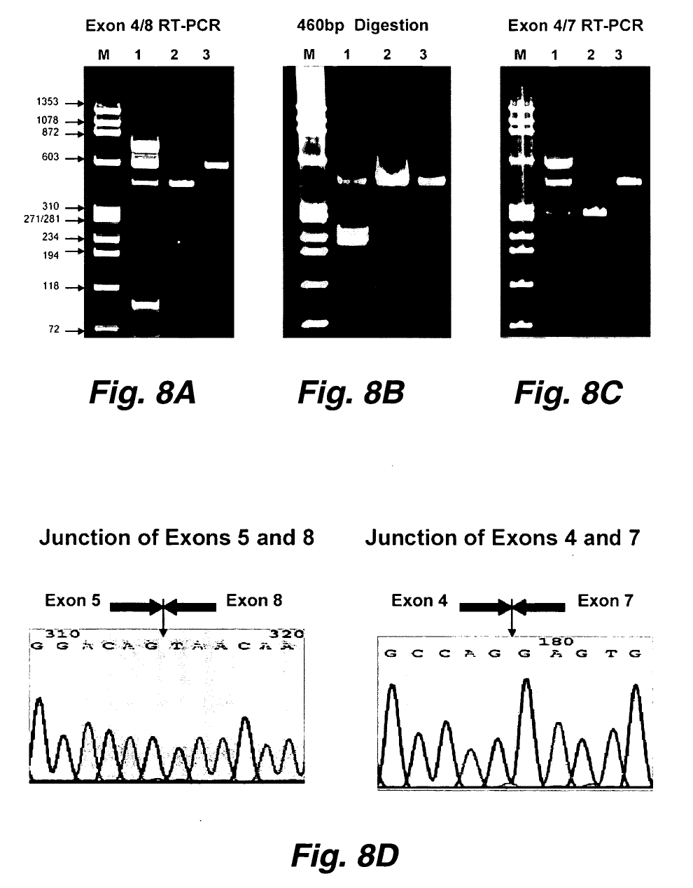

Since Ser phosphorylation of ERα in a ligand-independent manner has been described and implicated in the activation of ERα (4-8), we reasoned that any differential ERα phosphorylation patterns elicited by a redox stressing quinone relative to that of E2 might reflect differential ERα activities. Using the high-salt extracted and chromatin/nuclear matrix fractions isolated from MCF7 nuclei and shown in FIG. 7, Western blots were probed using an ERα antibody specific for Ser-118 phosphorylation (p-Ser-118). As shown in FIG. 9A, while both nuclear fractions after E2 treatment contained 67 kD p-Ser-118 ERα, the chromatin/nuclear matrix (pellet) fraction from K3 treated cells contained both 67 kD and 52 kD p-Ser-118 ERα with no ERα-specific signal detected in the highsalt nuclear fraction from K3 treated cells. While untreated (control) extracts showed no detectable 67 kD p-Ser-118 ERα, the chromatin/nuclear matrix (pellet) extracts from control and E2 treated cells contained low but readily detectable 52 kD p-Ser-118 ERα immunoreactivity.

As K3 has been shown to activate the MAPK pathway, nuclear extracts from MCF7 cells treated as described earlier but in the presence or absence of the MAPK inhibitor U0126 were examined for p-Ser-118 ERα. As shown in FIG. 9B, nuclear extracts from E2 treated cells show that the 67 kD p-Ser-118 ERα is unaffected by U0126 treatment, while K3 induced p-Ser-118 67 kD and 52 kD ERα are largely inhibited by U0126. Evaluating activation of MAPK under these various treatment conditions, FIG. 9B shows a high level of ERK1/ERK2 phosphorylation induced within 30 minutes of K3 treatment, with no phospho-ERK1/ERK2 detected at this same time point following E2 treatment, and only minimal activation detectable after co-treatment of cells with K3 and U0126. FIG. 9C extends these same observations of MAPK-dependent K3 induction of p-Ser-118 67 kD and 52 kD ERα to the ERα-positive human breast cancer cells, T47D, treated identically as the MCF7 cells.

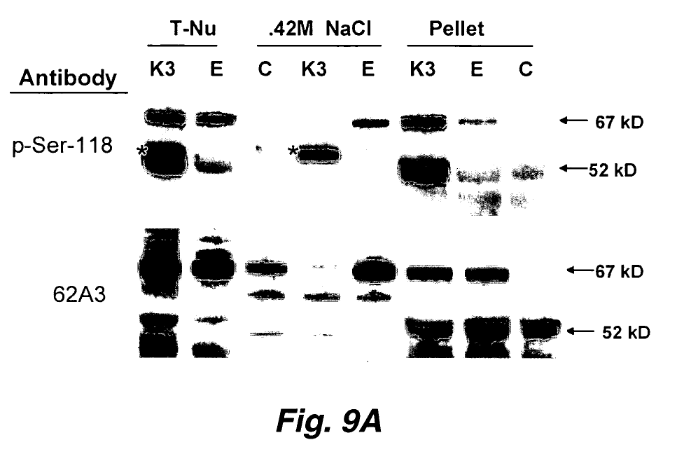

As Ser-167 phosphorylation of ERα has also been reported, a Western blot of nuclear extracts from MCF7 cells treated with K3 or E2 in the presence or absence of U0126 was probed with an antibody specific for p-Ser-167 ERα. As shown in FIG. 10, K3induces MAPK-dependent Ser-167 phosphorylation only on the 52 kD ERα nuclear variant, with no p-Ser-167 67 kD ERα detected. As well, p-Ser-167 ERα was not detected after E2 treatment, consistent with previous observations (9, 10). However, as seen with the p-Ser-118 52 kD variant, a low constitutative level of p-Ser-167 52 kD ERα is observed under control, K3+U0126, and E2 treatment conditions. Additionally, we found no affect on K3 induction of p-Ser-118 or p-Ser-167 ERα by co-treatment with either the PI-3 kinase inhibitor, LY294002, the p38 kinase inhibitor, SB203580, or the JNK inhibitor, SP600125 (data not shown).

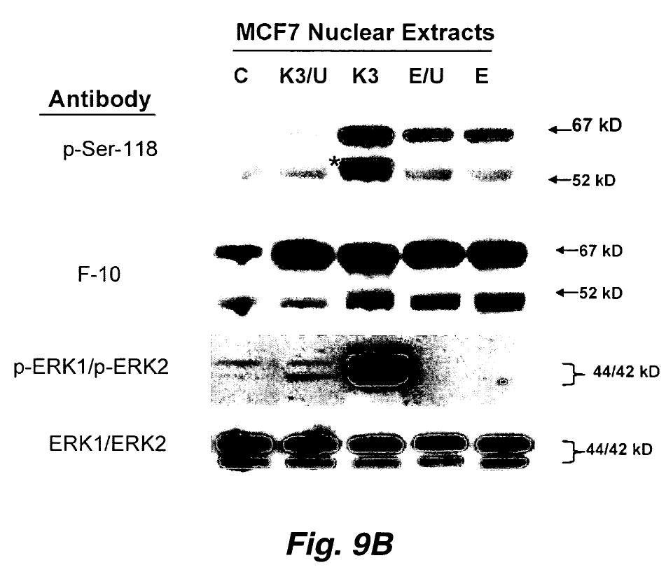

As a redox cycling and arylating quinone, K3 can either arylate nucleophilic substrates such as thiols or produce intracellular ROS (25, 28). To assess the role of K3 arylation on ERα phosphorylation, MCF7 cells were concurrently treated with a 100-fold molar excess of N-acetyl cysteine (NAC), a cell permeable thiol capable of quenching the arylating capacity of K3 (26). As FIG. 11A shows, concurrent NAC treatment blocked K3-induced p-Ser-118 and p-Ser-167 ERα formation, as well as MAPK activation as measured by phosphoERK1/2 formation, without affecting E2-induced p-Ser-118 67 kD ERα formation. Similarly, a specific inhibitor of NADPH: quinone oxidoreductase (NQO1), dicumarol (28,25), was added concurrently with K3 treatment. In agreement with a previous report and as shown in FIG. 11B, dicumarol has no impact upon K3 induced phospho-ERK1/2 formation (25), but produced at least 75% inhibition of K3-induced p-Ser-118 ERα and virtually complete inhibition of K3-induced p-Ser167 ERα formation. In contrast, dicumarol had no impact on E2-induced p-Ser118 ERα formation. Thus, while K3-induced ERα phosphorylation at either Ser118 or Ser-167 appeared dependent on an activated MAPK pathway, disruption of NQO1 mediated reduction of K3 to its dihydroquinone form by dicumarol overrides this MAPK activation and inhibits K3-induced ERα phosphorylations.

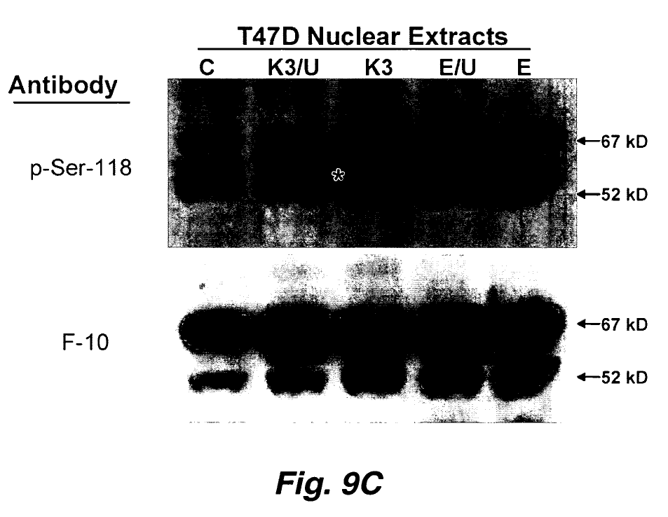

Extending these observations to ERα-positive breast cancer samples (T1, T2, T3), tumor lysates were prepared by high-salt (0.42M NaCl) vs. chromatin/nuclear matrix (pellet) extractions and immunoblotted for total ERα or p-Ser-118 ERα. As shown in FIG. 12, while these representative tumors contain abundant 67 kD ERα in lysates prepared by high-salt extraction protocols routinely used to quantitate breast tumor ERα, these same lysates showed little evidence of endogenous p-Ser-118 ERα formation and minimal evidence for 52 kD ERα variant expression. However, when the solubilized chromatin/nuclear matrix (pellet) fractions from these same tumors were analyzed, there was not only evidence for both 67 kD and 52 kD ERα expression, but there was pronounced evidence for endogenous p-Ser-118 52 kD ERα formation.

Discussion

Two ERα-positive human breast cancer cell lines, MCF7 and T47D, were used to study ERα responses induced by extracellular exposure to the protein arylating and redox-stressing quinone, K3. Following 30 minutes of exposure under estrogen-free culture conditions, K3 was found to induce nuclear translocation of cytoplasmic 67 kD ERα with concurrent activation of the MAPK pathway and induction of Ser-118 ERα phosphorylation. Although E2 treatment of these cells induces similar translocation and phosphorylation of 67 kD ERα, K3 treatment renders the phosphorylated full-length receptor much more resistant to routinely employed salt extraction methods, suggesting that K3 and E2 activation direct ERα translocation into separate nuclear compartments. While the biological importance of this differential nuclear compartmentalization is unclear, it may be associated with different cellular responses given the mitogenic role of E2 and the stress-promoting actions of K3.

The detection of a constitutively nuclear 52 kD ERα variant protein tightly associated with the chromatin/nuclear matrix fraction, and capable of undergoing rapid MAPK-dependent Ser-118 and Ser-167 phosphorylation in response to K3, represents the first report to our knowledge of an in vivo activated ERα variant. Previous studies have described Ser-167 phosphorylation of full-length ERα mediated by activation of the MAPK pathway (7,8); thus, it is surprising that we detected Ser-167 phosphorylation only in the 52 kD nuclear variant in response to a MAPK activating treatment with K3. Perhaps the nuclear compartmentalization and chromatin/nuclear matrix binding of this 52 kD ERα distinguishes it from saltextractable 67 kD ERα as a substrate for K3-induced Ser-167 phosphorylation. Although the functional implications of the different Ser-118 and Ser-167 phosphorylation responses to K3 between 67 kD ERα and the 52 kD nuclear variant are still unclear, it is possible that these different phosphorylation patterns represent signature responses to different types of cell stimulae which may prove diagnostically useful.

At the transcript level, a spectrum of exon-deleted ERα splice variants has been described (11-21); however, we are unaware of any reported splice variants capable of producing an ERα protein consistent with the size and epitope features described here for the 52 kD nuclear variant. Two studies putatively identified endogenous tumor cell (MCF7 and endometrial adenocarcinomas) expression of 52 kD ERα variants as exon-7 splice variants (24, 29). However, the frame-shift caused by such a single exon-7 deletion results in a truncated receptor lacking wildtype C-terminal epitopes, and thus would not be recognized by C-terminal specific ER antibodies like F-10 or D75 which clearly recognize the 52 kD nuclear variant reported here (FIGS. 6, 7). Since protein and epitope detection by an antibody used for immunoblotting depends on many factors (including extract preparation), it may be difficult to gage the intracellular protein abundance of the 52 kD nuclear variant relative to endogenous 67 kD ERα by immunoblotting. The fact that different antibodies can convey different protein abundances is illustrated by our immunoblot comparisons using the exon-4 specific SRA-1000 antibody vs. the N-terminal specific 62A3 antibody (FIGS. 6, 7), in which opposite proportions of 52 kD to 67 kD ERα within the same total nuclear extracts are suggested.

To be consistent with our immunoblotting results, splice variant transcripts encoding the observed 52 kD nuclear ERα must preserve the wildtype reading frame of the epitope-encoding exons 1, 2, 4 and 8, yet also lack sufficient coding sequence to result in the observed molecular wieght reduction. In the scheme of splicing variants generated by precise exon deletions, two possibilities capable of satisfying these conditions exist: deletion of exons 5 and 6 and deletion of exons 6 and 7. An RT-PCR search of MCF7 RNA detected both of these dual deletions involving exon 6 (FIG. 8) in addition to the previously described double exon 5 and 7 deletion and the single exon 5 and exon 7 deletions (17). Identifying which of these dual deletions involving exon 6 results in the nuclear 52 kD ERα variant expressed in MCF7 and T47D breast cancer cells is now under a more comprehensive proteomics investigation, as each would produce unique peptide fragments resulting from the juxtaposition of exon 4 with 7 or exon 5 with 8. Given the rather broad width of the observed 52 kD ERα immunoblot band, it is possible that both splice variants are expressed and their products similarly sequestered within the nucleus of these cells. The extremely weak immunoreactivity of this band on immunoblotting with the H222 monoclonal (which has a complex epitope specificity that primarily spans exon-7 encoded residues) as well as molecular size considerations suggest that the 52 kD nuclear ERα protein most likely arises from the splice variant transcript missing exons 6and7.

The possibility that a low abundance ERα splice variant can produce what appears on immunoblotting to be a relatively high abundance of the nuclear 52 kD variant receptor is supported by preliminary protein half-life observations and potential mechanisms known to regulate ERα translation efficiency. ERα half-life estimates were performed by treating estrogen-free MCF7 cultures with E2+/cycloheximide (data not shown). While nuclear 67 kD ERα levels decayed with an expected ˜3 h half-life, there was no detectable decline in the nuclear 52 kD ERα protein following 3 h of combined E2 and cycloheximide treatment relative to E2 alone. Since E2 treatment alone is known to decrease the half-life of 67 kD ERα (30), the absence of an intact LBD in the 52 kD ERα variant probably contributes to its nuclear stability. Recent reports have also revealed the importance of 5′ untranslated regions (UTR) within ERα transcripts that determine the translation efficiency and rate of intracellular ERα protein production (31).

Mechanisms regulating ERα splicing may be linked to the same ERα promoter-choosing mechanisms known to direct the spectrum of different 5′ UTRs that regulate ERα translation. Thus, an attractive and testable hypothesis is that a very stable 52 kD nuclear receptor variant is encoded by an alternatively spliced ERα mRNA of low abundance bearing a specific 5′ UTR sequence that directs its translation with high efficiency.

Another novel observation emerging from this study was the unexpected dependence of K3 activated Ser-118 and Ser-167 ERα phosphorylation on the relatively ubiquitous NAPDH quinone oxidoreductase-1 (NQO1), and its prevention by the specific NQO1 inhibitor dicumarol. In agreement with a previous report (25, 32), we found that dicumarol has no influence on the demonstrated ability of K3 to activate the U0126-sensitive MAPK pathway that phosphorylates 44/42 kD ERK1/2. K3 activation of the MAPK pathway is thought to result from this quinone's ability to undergo Michael addition reactions and arylate key Cys residues within the catalytic domain of phosphatases, thereby inactivating negative regulators of growth-promoting receptors that signal through the MAPK pathway (25,26). The ability of the cell-permeable thiol donor, Nacetyl cysteine (NAC), to completely block K3 phosphorylation of ERK1/2 supports the mechanistic role of quinone arylation in mediating this effect. It might also be concluded that K3 activation of ERα phosphorylation at Ser-118 and Ser-167 depends entirely on this arylating reactivity, since K3 induction of ERα phosphorylation can be completely prevented by co-treatment with the MEK inhibitor U0126. However, the ability of dicumarol co-treatment to prevent K3 induced ERα phosphorylation indicates that this form of ligand-independent ERα activation is also dependent upon cellular NQO1 activity, and the probable requirement for NQ01-mediated two electron conversion of K3 to its dihydroquinone form, in addition to MAPK pathway activation. Interestingly, other studies have implicated dicumarol as a negative regular of such stress kinases as JNK and p38 (32, 33).

While further studies are needed to define specific components of these seemingly unrelated MAPK and NQO1 pathways that mediate K3 activation of ERα, the potential requirement for K3 conversion to its dihydroquinone form suggests that other more biologically relevant dihydroquinones, which can be oxidized intracellularly to their quinone forms, might also induce ligandindependent ERα activation and Ser phosphorylation. To explore this possibility, we treated MCF7 cells with the dihydroquinone form of the endogenously produced estrogen catechol, 4-hydroxyestrone (4-OHE). In addition to activating the MAPK pathway and phosphorylating ERK1/2, 4-OHE induced Ser-118 and Ser-167 phosphorylation of the 52 kD ERα nuclear variant, albeit at lower levels than that induced by K3 treatment (data not shown).

In conclusion, we have demonstrated that a constitutive and nuclear-bound 52 kD variant with N-terminal, exon4 and C-terminal epitopes of ERα becomes rapidly phosphorylated at Ser-118 and Ser-167 residues in the absence of any estrogenic ligand and in response to the arylating and redox-active quinone, K3. This ligand-independent K3 induction of ERα phosphorylation appears dependent on two signaling pathways, MAPK and NQO1, which have as yet no reported links to one another. As well, a two-step extraction of several ERα positive breast cancer samples reveals the presence of this Ser-118 phosporylated 52 kD ERα nuclear variant within a subcellular compartment that would likely escape detection if analyzed by routine high-salt extraction protocols typically used to quantitate wildtype 67 kD ERα.

Materials and Methods

Cell Lines and Cell Treatment Conditions.

Human breast cancer cell lines MCF7 and T47D were obtained from the American Type Culture Collection (ATCC). MCF7 cells were maintained in Dulbecco's Modification of Eagle's Medium (Cellgro) supplemented with 10% fetal bovine serum (Cellgro), 1% penicillin/streptomycin (Cellgro) and 10 μg/ml insulin (Sigma). T47D cells were maintained in RPMI (Cellgro) supplemented with 10% fetal bovine serum, 1% penicillin/streptomycin and 10 μg/ml insulin. Treatment conditions for both cell lines involved plating ˜1×106 cells in normal media onto 10 cm dishes, attachment and growth for 24 hours followed by a change to estrogen-free culture conditions (phenol red-free DME H-16 supplemented with 10% charcoal stripped serum, 1% penicillin/streptomycin and 10 μg/ml insulin) for an additional 24 hours. Cells were then treated as indicated for 30 minutes before extract preparation. Indicated conditions involved 30 minute pretreatments.

Reagents.

The ERα antibodies used in this study include F-10 (Santa Cruz Biotechnology), H222 (Lab Vision), D75 (Lab Vision), 62A3 (Cell Signaling), SRA1000 (Stessgen), Ab-8 (Lab Vision), Ab-6 (Lab Vision), the 16JR monoclonal antibody to phosphorylated Ser-118 (Cell Signaling) and the rabbit polyclonal antibody to phosphorylated Ser-167 (sc-12955-R, Santa Cruz Biotechnology). HRP coupled goat anti-rat was obtained from Santa Cruz Biotechnology, HRP coupled goat anti-mouse and HRP coupled goat anti-rabbit were obtained from BioRad. U0126, LY294002 and SB203580 were from Calbiochem; SP600125 was from A. G. Scientific, N-acetyl cysteine and dicumarol were from Sigma.

Immunofluorescence.

Cells were plated, grown and treed as described above on Lab-Tek II Chamber Slides (Nalge Nunc). Cells were fixed with 4% paraformaldehyde (Sigma), permeabilized with 0.5% trifion X-100 (Sigma) and blocked with 5% normal goat serum (Rockland Biochemicals) for 1 hour in wash buffer (10 mM Tris pH 7.5, 150 mM NaCl, 1% BSA). Fixed, permeabilized and blocked cells were first incubated with ERα antibody F-10 (1:250 dilution) for 1 hour at room temperature in wash buffer containing 2.5% goat serum, and then incubated for 1 hour at room temperature with a fluorescently conjugated goat anti-mouse secondary (Molecular Probes) in the wash buffer containing 2.5% goat serum. DNA was visualized by addition of DAPI (0.5 μg/ml) into the wash, and slides were mounted (Vector Laboratories) and viewed and photographed using a Nikon E800 upright fluorescence microscope.

Nuclear Fractionations.

Following aspiration of the media and one ice cold PBS wash, cells growing on 10 cm dishes were harvested using a cell scraper and 0.7 ml of ice cold cell lyses buffer (10 nM HEPES pH 7.9, 1.5 mM MgCl2, 10 nM KCl, 1 mM DTT, 10 nM NaFl, 5% glycerol, 0.45% NP40 (Igepal, Sigma) and mincomplete protease inhibitors (Roche)). Nuclei were pelleted at 4 oC in a microcentrifuge set at 3,000×g for 4 minutes. For total nuclear extracts, nuclei were resuspended in 90 μl of DNase I digestion buffer (20 mM Tris pH 7.5, 100 nM NaCl and 10 mM MgCl₂) and incubated with 300 units of DNase I at room temperature for 5 minutes. Following the DNase I digestion, complete solublization of the nuclei was achieved by the addition of SDS to 1% to the DNase I digestion reaction. For preparation of high-salt nuclear extracts (nucleoplasm), isolated nuclei were resuspended for 20 minutes in ice cold extraction buffer (0.4 M NaCl, 25 mM Tris pH 7.5, 1 mM DTT, 20% glycerol, 10 mM NaFl and min-complete protease inhibitors (Roche)) followed by a 4° C. centrifugation at 16,000×g for 15 minutes. The resulting supernatant comprised the high-salt nuclear fraction while the pelleted material solublized by DNase I digestion and addition of SDS comprised the chromatin/nuclear matrix containing fraction.

Western Blotting.

Equal amounts of protein from the various extracts were mixed with 2× SDS sample buffer (125 mM Tris pH6.8, 20% glycerol, 2% SDS, 0.28 M 2-mercaptoethanol and0.5% Bromphenol Blue), heated for 5 minutes at 90° C. and electrophoresed on NuPAGE 4-12% Bis-Tris gels (Invitrogen) using NuPAGE MOPS SDS running buffer (Invitrogen) and full range Rainbow recombinant protein molecular weight markers (Amersham Pharmacia). Gels were electroblotted onto Hybond ECL nitrocellulose membranes (Amersham Pharmacia) in standard transfer buffer (25 mM Tris, 200 mM glycine with 20% methanol) at 250 milliamps for 1 hour at room temperature. Membranes were then blocked for 30 minutes in blocking buffer (150 mM NaCl, 20 mM Tris pH 7.5, 0.3% Tween-20 and 4% Nonfat dry milk powerby weight). Following blocking, membranes were incubated overnight at 4° C. with the primary antibody in blocking buffer using a 1:1000 dilution of the supplied antibody concentration. Membranes were then washed three times for 5 minutes each in blocking buffer without the milk power, incubated with a HRP conjugated secondary antibody at 1:10,000 dilution in blocking buffer for 1 hour at room temperature, and washed three times for 10 minutes each in blocking buffer without dry milk power. Membranes were then developed using SuperSignal West Pico Chemiluminescent substrate (Pierce) as per manufacture's instructions.

RT-PCR and ERα Primers.

The exon-4 forward primer, 5′-ctcatgatcaaacgctctaag-3′ (SEQ ID NO: 1), and the exon-8 reverse primer, 5′-acggctagtgggcgcatgta-3′ (SEQ ID NO:2), were used as previously described (17). The exon-7 reverse primer was 5′-catcaggtggatcaaagtgtctg-3′ (SEQ ID NO:3). Total RNA was prepared from MCF7 cells growing in normal media or charcoal stripped serum using TRIzol (Invitrogen) according to the manufacture's instructions. 2 μg of total RNA from cells grown under normal and charcoal stripped culture conditions was primed with oligo dT and reversed transcribed (RT) in 20 μl volumes using SuperScript II RNase H-Reverse Transcriptase (Invitrogen) with reaction protocols and buffers supplied by the manufacture. PCR was preformed in a 50 μl volume using 2 μl of the RT reaction, 0.5 μM of each ERα primer, Pfu Turbo DNA polymerase and the manufacturer's (Strategene) reaction buffer. Thermocycling consisted of 32 cycles of 30 seconds at 95° C., 30 seconds at 56° C., and 30 seconds at 72° C. PCR products were analyzed on 8% polyacrylamide gels (Invitrogen) using a 1×Tris/Borate/EDTA running buffer with ΦX Hae III digested DNA for markers and staining in ethidium bromide to visualize DNA. RT of RNA from cells grown in normal vs. estrogen-free culture conditions produced no qualitative differences in the resulting profile of PCR products.

### REFERENCES

1. Mangelsdorf D J, Thummel C, Beato M, Herrlich P, Schutz G, Umesono K, Blumberg B, Kastner P, Mark M, Chambon P, Evans R M 1995 The nuclear receptor superfamily: the second decade. Cell 83:835-839.

2. Tsai M-J, O'Malley B W 1994 Molecular mechanisms of action steroid/thyroid receptor superfamily members. Annu Rev Biochem 63:451-486ERα.

3. Beato M, Sanchez-Pacheco A 1996 Interaction of steroid hormone receptors with the transcription initiation complex. Endocr Rev 17:587609.

4. Kato S, Endoh H, Masuhiro Y, Kitamoto T, Uchiyama S, Sasaki H, Masushige S, Gotoh Y, Nishida E, Kawashima H, Metzger D, Chambon P 1995 Activation of the estrogen receptor through phosphorylation by mitogen-activated protein kinase. Science 270:1491-1494.

5. Joel P B, Traish A M, Lannigan D A 1995 Estradiol and phorbol ester cause phosphorylation of serine 118 in the human estrogen receptor. Mol Endrocrinol 9:1041-1052.

6. Bunone G, Briand P A, Miksicek R J, Picard D 1996 Activation of the unliganded estrogen receptor by EGF involves the MAP kinase pathway and direct phosphorylation. EMBO 15:217421-83.

7. Joel P B, Smith J, Sturgill T W, Fisher T L, Blenis J, Lannigan D A 1998 pp90rsk1 regulates estrogen receptor-mediated transcription through phosphorylation of Ser-167. Mol Cell Biol 18:1978-1984

8. Lannigan D A 2003 Estrogen receptor phosphorylation Steroids 68:1-9

9.Le Goff P, Montano M M, Schodin D J, Katzenellbogen B S 1994 Phosphorylation of the human estrogen receptor. Identification of hormone sites and examination of their influence on transcriptional activity. J Biol Chem 269:4458-4466.

10. Joel P B, Traish A M, Lannigan 1998 Estradiol-induced phosphorylation of Serine 118 in the estrogen receptor is independent of p42/p44 mitogen-activated protein kinase J Biol Chem 273:13317-13323.

11. Fuqua S A, Fitzgerald S D, Chamness G C, Tandon A K, McDonnell D P, Nawaz Z, O'Mally B W, McGuire W L 1991 Variant human breast tumor estrogen receptor with constitutive transcriptional activity. Cancer Res 51:105-109.

12. Castles C G, Fuqua S A, Klotz D M, Hill S M 1993 Expression of a constitutively active estrogen receptor variant in the estrogen receptornegative BT-20 human breast cancer cell line. Cancer Res 53:59345939.

13. Miksicek R J, Lei Y, Wang Y 1993 Exon skipping gives rise to alternatively spliced forms of the estrogen receptor in breast tumor cells. Breast Cancer Res Treat 26:163-179.

14. Castles C G, Klotz D M, Fuqua S A, Hill S M 1995 Coexpression of wildtype and variant oestrogen receptor mRNA in a panel of human breast cancer cell lines. Br J Cancer 71:974-980.

15. Gotteland M, Desauty G, Delarue J C, Liu L, May E 1995 Human estrogen receptor messenger RNA variants in both normal and tumor breast tissues. Mol Cell Endocrinol 112:1-13.

16. Fuqua S A, Wolf D M 1995 Molecular aspects of estrogen receptor variants in breast cancer. Breast Cancer Res Treat 35:233-241.

17. Zhang Q X, Hilsenbeck S G, Fuqua S A, Borg A 1996 Multiple Splicing Variants of the Estrogen Receptor are Present in Individual Human Breast Tumors. J Steroid Biochem Mol Biol 59 (3-4):251-260.

18. Murphy L C, Leygue E, Dotzlaw H, Douglas D, Coutts A, Watson P H 1997 Oestrogen receptor variant and mutations in human breast cancer. Ann Med 29:221-234.

19. Hopp T A, Fuqua S A 1998 Estrogen receptor variants. J Mammary Gland Biol Neoplasia 3:73-83.

20. Poola I, Speirs V 2001 Expression of alternatively spliced estrogen receptor alpha mRNAs is increases in breast cancer tissue. 2001 J Steroid Biochem Mol Biol 78:459-469.

21. Poola I, Koduri S, Chatra S, Clarke R 2000 Identification of twenty alternatively spliced estrogen receptor alpha mRNAs in breast cancer cell lines and tumors using splice targeted primer approach. 2000 J Steroid Biochem Mol Biol 72:249-258.

22. Pedrero G, Zuazua J M, Martinez-Campa C, Lazo P S, Ramos S 2003 The naturally occurring variant of estrogen receptor (ER) ERDeltaE7 suppresses estrogen-dependent transcriptional activation by both wildtype ERalpha and ERbeta Endocrinol 144:2967-2976.

23. Bollig A, Miksicek R J 2000 An Estrogen Receptor-˜Splicing Variant Mediates Both Positive and Negative Effects on Gene Transcription. Mol Endocrinol 14:634-649.

24. Horvath G, Leser G, Helou K, Henriksson M 2002 Function of the Exon 7 Deletion Variant Estrogen Receptor˜Protein in an Estradiol Resistant, Tamoxifen-Sensitive Human Endometrial Adenocarcinoma Grown in Nude Mice. Gynecologic Oncology 84:271-279.

25. Klotz L O, Patak P, Ale-Agha N, Buchczyk D P, Abdelmohsen K, Gerber P A, von Montfort C, Sies H 2002 2-Methyl-1,4naphthoquinone, Vitamine K3, Decreases Gap-Junctional Intercellular Communication via Activation of the Epidermal Growth Factor Receptor/Extracellular Signal-regulated Kinase Cascade. Cancer Res 62:4922-4928.

26. Abdelmoshsen K, Gerber P A, von Montfort C, Sies H, Klotz L O 2003 Epidermal growth factor receptor is a common mediator of quinoneinduced signaling leading to phosphorylation of connexin-43: role of glutathione and tyrosine phosphatases. J Biol Chem 278:38360-38367.

27. Liang X, Lu B, Scott G K, Chang C-H, Baldwin M A, Benz C C 1998 Oxidant stress impaired DNA-binding of estrogen receptor from human breast cancer. Mol Cell Endocrinol 146:151-161.

28. Cullen J J, Hinkhouse M M, Grady M, Gaut A W, Liu J, Zhang Y P, Weydert C D, Domann F E, Oberley L W 2003 Dicumarol Inhibition of NADPH: Quinone Oxidoreductase Induces Growth Inhibition of Pancreatic Cancer via a Superoxide-mediated Mechanism. Cancer Res 63:5513-5520.

29. Fasco M J, Keyomarsi K, Arcaro K F, Gierthy J F 2000 Expression of an estrogen receptor alpha variant protein in cell lines and tumors. Mol Cell Endocrinol 162:167-180.

30. Nawaz Z, Lonard D M, Dennis A P, Smith C L O'Malley B W 1999 Proteasome-dependent degradation of the human estrogen receptor. Proc Natl Acad Sci USA 96:1858-1862.

31. Kos M, Denger S, Reid G, Gannon F 2002 Upstream Open Reading Frames Regulate the Translation of the Multiple mRNA Variant of the Estrogen Receptor α. J Biol Chem 277:37131-37138.

32. Cross J V, Deak J, Rich E A, Qian Y, Lewis M, Parrott L A, Mochida K, Gustafson D, Pol S V, Templeton D J 1999 Quinone Reductase Inhibitors Block SAPK/JNK and NF˜B Pathways and Potentiate Apoptosis. J Biol Chem 274:31150-31154.

33. Seanor K L, Cross J V, Nguyen S M, Yan M, Templeton D J 2003 Reactive Quinones Diffefrentially Regulated SAPK/JNK and p38/mHOG Stress Kinases. Antioxidants and Redox Signaling 5:103113.

### Example 4

### Activation of Nuclear Factor-κB (NFκB) Identifies a High-Risk Subset of Hormone-Dependent Breast Cancers

Activation of nuclear factor-|B (NFκB) has been linked to the development of hormoneindependent, estrogen receptor (ER)-negative human breast cancers. To explore the possibility that activated NF|B marks a subset of clinically more aggressive ER-positive breast cancers, NFκB DNA-binding was measured in ER-positive breast cancer cell lines and primary breast cancer extracts by electrophoretic mobility shift assay and ELISA-based quantification of specific NF|B p50 and p65 DNA-binding subunits. Oxidant (menadione 100∝M×30 min) activation of NFκB was prevented by pretreatment with various NFκB inhibitors, including the specific NFκB kinase (IKK) inhibitor, parthenolide (PA), which was found to sensitize MCF-7/HER2 and BT474 but not MCF-7 cells to the antiestrogen tamoxifen. Early stage primary breast cancers selected a priori for lower ER content (21-87 fmol/mg; n=59) and known clinical outcome showed 2-4 fold increased p50 and p65 NF| B DNA-binding over a second set of primary breast cancers with higher ER content (>100 fmol/mg; n=22). Breast cancers destined to relapse (13/59) showed significantly higher NFκB p50 (but not p65) DNA-binding over those not destined to relapse (46/59; p=0.04). NFκB p50 DNA-binding correlated positively with several prognostic biomarkers; however, only NFκB p50 DNA-binding (p=0.04), Activator Protein-1 DNA-binding (AP-1; p<0.01) and urokinase-type plasminogen activator expression (uPA; p=0.0014) showed significant associations with metastatic relapse and disease-free patient survival. These clinical findings indicate that high-risk ER-positive breast cancers may be prognostically identified by increased NF| B p50 DNA-binding, and support preclinical models suggesting that therapeutic inhibition of NFκB activation may improve the endocrine responsiveness of high-risk ER-positive breast cancers.

Introduction

Nuclear factor-κB (NFκB) is a family of ubiquitously expressed transcription factors that for nearly two decades has been known to be redox-sensitive and to regulate immune and inflammatory responses (Allen and Tresini, 2000; Baeuerle and Baltimore, 1996; Ghosh et al., 1998). Today, NFκB is generally recognized as a key cellular mediator acting “at the crossroads of life and death” (Karin and Lin, 2002). Indeed, NFκB activation in response to extracellular chemical stresses, various cytokines and growth factor ligands directly regulates at least 150 target genes whose cellular influences extend well beyond those of the immune system (Pahl, 1999). The anti-apoptotic, proliferation, motility and invasion promoting roles of NFκB appear to be essential for normal organ development and may be disturbed with organ aging. NFκB also becomes constitutively overactive during progression of various chronic inflammatory disorders and malignancies such as B and T cell lymphomas and leukemias, thyroid, head and neck, gastrointestinal, and breast carcinomas (Baldwin, 2001; Feinman et al., 2004; Giardina and Hubbard, 2002; Veiby and Read; 2004). The observed constitutive activation of NFκB in such a broad array of pathophysiological disorders supports a prevalent belief that the NFκB pathway is a clinically relevant and mechanistically important target for inhibition by new drugs currently under development (Feinman et al., 2004; Ghosh and Karin, 2002; Karin et al., 2004; Veiby and Read, 2004; Yamamoto and Gaynor, 2001).

The NFκB family consists of five mammalian members: p50 (NFκB1), p52 (NFκB2), p65 (relA), c-rel, and relB. These all share a conserved 300 amino acid Nterminal Rel homology domain (homologous to that encoded by the avian oncogene, v-Rel) that is responsible for dimerization, nuclear translocation, DNA-binding, and association with IκB inhibitory proteins (Dixit and Mak, 2002; Ghosh and Karin, 2002).

These Rel family members exist as homo- or heterodimers, although the most abundant form of intracellular NFκB is generally acknowledged to be the p50/p65 heterodimer. In resting cells NFκB is cytoplasmically sequestered as latent forms bound to one or more members of the IκB protein family (IκBΕ, IκBβ, IκBε, IκBγ, Bcl-3, and the precursor Rel proteins p100 and p105). Various cell stimuli (e.g., TNFα, CD40 ligand, IL-1, LPS, TRANCE, EGF, phorbol esters, peroxides, ionizing radiation) induce cytoplasmic phosphorylation (via activation of the IκB kinase complex, IKK) and subsequent proteasomal degradation of IκB inhibitory proteins, activating NFκB for translocation into the nucleus where it binds promoter-specific κB consensus elements and regulates the transcription of NFκB-dependent genes. While phosphorylation and degradation of IκB inhibitory proteins are considered the rate-limiting if not obligate mechanisms by which NFκB is activated, novel IKK-independent pathways leading to IκB proteasomal degradation as well as NFκB phosphorylating kinases are now known that can also activate NFκB. Most activated forms of NFκB stimulate gene transcription, although specific NFκB subunits lack transactivation domains; thus, activation and nuclear translocation of p50/p50 and p52/p52 homodimers result in repression of NFκB-dependent genes (Ghosh and Karin, 2002). Curiously, when either the NFκB p50 or p52 products of the p105 and p100 Rel precursor proteins are bound to the oncogenic and noninhibitory IκB family member, Bcl-3, they become transcriptionally competent and stimulate expression of NFκB-dependent genes (Cogswell et al., 2000; Ghosh and Karin, 2002). Among experimental and medicinal strategies to inhibit constitutively active NFκB are drugs that target upstream signaling mediators or downstream IκB degradative mechanisms (Yamamoto and Gaynor, 2001), including the potent and specific antioxidant pyrrolidine dithiocarbamate (Schreck et al., 1992), proteasome inhibitors like MG-132 and PS-341 (bortezomib/Velcade) (Feinman et al., 2004), or 5 sesquiterpene lactones found in antiphlogistic plant extracts like the specific IKK inhibitor, parthenolide (PA) (Hehner et al., 1999).

We became interested in the role of NFκB activation in the development and progression of hormone-dependent, estrogen receptor-α (ER) overexpressing breast cancers with recognition that these are a clinically and biologically diverse group of breast cancers associated with age-dependent activation of oxidant stress pathways (Benz, 2004; Eppenberger-Castori et al., 2002; Quong et al., 2002). NFκB activation is now known to be absolutely required for normal mammary gland development; and, as recently reviewed (Cao and Karin, 2003), the constitutive activation of NFκB has been linked with the etiology and progression of hormone-independent (ER-negative) breast cancers, in part due to its transcriptional stimulation of genes that direct cell proliferation and invasion such as cyclin D1 and urokinase-type plasminogen activator (uPA). When first evaluated by DNA-binding, transactivation and immunoblot assays, NFκB activation was reported to be aberrant in a subset of human breast cancers (Sovak et al., 1997), minimal in ER-positive cancers and cell lines, yet constitutively elevated in ER-negative cancers and cell lines (Nakshatri et al., 1997). A subsequent study compared a limited number of breast cancers against normal adjacent breast tissue (and also against a panel of breast cancer cell lines) by measuring total NFκB DNA-binding activity and subunit (p65, c-rel, p52, p50) protein and transcript expression levels (Cogswell et al., 2000). Breast cancer samples all showed greater total NFκB DNA-binding activity than normal tissue counterparts, but this increased activity did not correlate with tumor ER status, unlike results comparing ER-positive vs. ER-negative breast cancer cell lines. While breast cancer cell lines showed predominantly increased p65 subunit expression and p65/p50 NFκB DNA-binding activity, breast tumor samples showed selective upregulation of p50, p52 and c-rel expression (as well as Bcl-3) and constitutively increased DNA-binding by complexes composed primarily of these subunits and relatively little p65, suggesting a different pattern of NFκB activation between breast cancer cell lines and tumor samples (Cogsell et al., 2000).

The present study was undertaken to clarify the extent and clinical importance of NFκB activation by studying a biologically diverse set of ER-positive breast cancer cell lines and primary breast tumor samples, collectively referred to as hormone-dependent human breast cancers. ER-positive breast cancer cells were shown to be capable of NFκB activation by oxidant stress; and endocrine-responsive vs. endocrine-resistant ER-positive cell lines were shown to be differentially responsive to the antiestrogensensitizing effects of NFκB inhibition. By utilizing a novel ELISA-based assay to quantitate specific p65 and p50 NFκB DNA-binding subunits, these specific activities were independently assessed in ER-positive breast tumor samples grouped by ER content, and evaluated for association with clinical outcome within a group of comparably staged breast cancers previously characterized by a large panel of other prognostic biomarkers.

Materials and Methods

Cell Lines, Treatments, Viability Determination and Subcellular Fractionation.

The ER-positive MCF-7 and BT474 human breast cancer cell lines were obtained from the American Type Culture Collection (Rockvile, Md.) and maintained at 37° C. and 5% CO2 in Dulbecco's modified Eagle's medium (DMEM) for MCF7 or RPMI-1640 medium for BT474, supplemented with 10% fetal bovine serum (FBS), 1% penicillin-streptomycin, and 10 μg/ml insulin. Media and supplements were purchased from Mediatech, Inc. (Herndon, Va.). The HER2/ErbB2 overexpressing MCF-7 subline, MCF-7/HER2 (clone-18), has been previously characterized and is maintained in supplemented media as described for parental MCF-7 cells, in addition to G418 selection (Benz et al., 1992).

Tamoxifen ([Z]-1-[p-dimethylaminoethoxyphenyl]-1,2-diphenyl-1 butene), PDTC (pyrrolidine dithiocarbamate), MG-132, and menadione (vitamine K3, 2-methyl-1,4-naphthoquinone) were purchased from Sigma Chemical Co. (St. Louis, Mo.); and parthenolide (PA) was purchased from Alexis Biochemicals (San Diego, Calif.). PS-341 was kindly provided by Millennium Pharmaceuticals Inc. (Cambridge, Mass.). For the assessment of NFκB inhibitors on menadione-induced oxidative stress, cells in nearconfluent cultures were pretreated with MG-132 (25 μM), PS-341 (5 μM), PDTC (100 μM), or PA (50 μM) for 30 min prior to the addition of menadione (100 μM), and cultures harvested 30 min later. For assessment of combined treatment effects on cell viability, 2×104 cells (MCF-7, MCF-7/HER2, BT474) grown in 24-well plates were first treated with tamoxifen (dissolved in ethanol) and/or parthenolide (dissolved in DMSO); for the combination treatment pathenolide was added 4 h before addition of tamoxifen. Cell viability was measured 18 h after tamoxifen treatment using the sulforhodamine B (SRB) assay (Skehan, et al. 1990). Briefly, cells fixed with trichloroacetic acid were stained for 30 min with 0.4% SRB dissolved in 1% acetic acid. Unbound dye was removed by four washes with 1% acetic acid, and protein-bound dye was extracted with 10 mM unbuffered Tris base [tris(hydroxymethyl)aminomethane] for determination of optical density at 564 nm.

Fractionation of control and treated cells into cytoplasmic and nuclear extracts was performed as previously described (Dignam et al., 1983), with minor modification. Cells harvested on ice were washed twice with cold PBS, scraped and resuspended in 1.0 ml hypotonic buffer (20 mM HEPES, pH 7.0; 10 mM KCl; 1 mM MgCl2; 0.1% Triton X-100; 20% glycerol; 0.5 mM DTT) containing a cocktail of protease inhibitors (Mini CompleteTM protease inhibitors, Roche Diagnostics, Mannheim, Germany). The harvested cells were Dounce homogenized on ice, the mixture centrifuged at 3,000 rpm at 4° C. (5 min), and the cytoplasmic fraction separated from the nuclear pellet which was resuspended in elution buffer (20 mM HEPES, pH 7.0; 10 mM KCl; 1 mM MgCl2; 0.42 M NaCl; 0.1% Triton X-100; 20% glycerol; 0.5 mM DTT), supplemented with protease inhibitors. After 20 min at 4° C. the buffered mixture was centrifuged at 14,000 rpm at 4° C. (10 min) to obtain solubilized nuclear extracts which were then stored in aliquots at −80° C. after protein determination (Bradford; BioRad, Hercules, Calif.) and for subsequent NFkB DNA-binding assays.

Human Breast Tumor Study Samples and Extracts.

Cryobanked (−80° C.) sample extracts, prepared from primary human breast tumors with known ER-positive receptor status (Liang et al., 1998; Quong et al., 2002), were subdivided into two study groups according to associated data and tumor ER content (quantitated by ER-EIA; Abbott Labs, IL): Group A tumors (n=22) with ER>100 fmol/mg extract protein; and Group B tumors (n=59) with ER=21-87 fmol/mg extract protein (median 47 fmol/mg). Group A tumors were unassociated with any clinical or other biomarker data, but were analyzed for Sp1 DNA-binding in addition to NFκB DNA-binding for this study. Group B tumors chosen for NFκB DNA-binding analysis in this study have been previously reported (Quong et al., 2002) and had been preselected as a group for their age range (37-76 years; median 54 years), uniform early tumor stage (pT1 or pT2; node-negative), known tamoxifen adjuvant treatment status and clinical follow-up for breast cancer relapse (52 month median follow-up interval), and previous biomarker analyses including AP-1 and Sp1 DNA-binding, ERα, PR, pS2, ErbB2, EGFR, phospho-Erk5, iNOS, cathepsin D and uPA content (Eppenberger et al., 1998; Eppenberger-Castori et al., 2001; Eppenberger-Castori et al., 2002; Quong et al., 2002).

All tumor extracts had been prepared in accordance with standard procedures for ERα and PR receptor determination. In brief, surgically excised, trimmed and snap frozen breast tumors were finely pulverized in liquid nitrogen using a Micro-Dismembrator U (B. Braun, Melsungen, Germany). The tumor powders were homogenized with a tissue homogenizer (Ultra-Turrax, Janke & Kunkel, Staufen, Germany) for 20 seconds in 3 volumes of ice-cold extraction buffer containing 10 mM Tris, 1.5 mM EDTA, 10% glycerol, 5 mM disodiummolybdate and 1 mM monothioglycerol. The homogenate was centrifuged for 3 minutes at 4° C. and the supernatant recentrifuged in an ultracentrifuge (Beckman Instruments, Fullerton, Calif.) at 100,000×g for 40 minutes at 4° C. The resulting supernatants (tumor extracts) were kept frozen in multivial aliquots at −80° C. until thawed for biomarker determination.

Immunoblots and NFκB DNA-Binding Assays.

Antibodies against p50 NFκB, p65 NFκB, IκB, and β-actin proteins were commercially obtained (Santa Cruz Biotechnology, Santa Cruz, Calif.; and Abcam Inc., Cambridge, Mass.). Tumor cell nuclear extracts (15 μg protein) were boiled in loading buffer (125 mM Tris-HCl, pH 6.8; 4% SDS; 20% glycerol; and 10% 2-mercaptoethanol) and resolved by electrophoresis in 4-12% Bis-Tris SDS gradient gels in morpholinepropane sulfonic acid (MOPS) buffer. Separated proteins were transferred onto polyvinylidene difluoride membranes (Millipore Co., Billerica, Mass.), blocked with 5% non-fat milk in PBS containing 0.1% Tween-20, and membranes immunoblotted with the indicated antibodies in blocking solution. Bound antibodies were visualized by the enhanced chemiluminescence reaction using horseradish-peroxidaseconjugated goat antibody against mouse or rabbit IgG (BioRad, Hercules, Calif.) and chemiluminescence enhancement reagents (Pierce, Rockford, Ill.).

NFκB DNA-binding activity was measured by two different techniques. Analogous to previous tumor extract scoring of Sp1 DNA-binding protein (Quong et al., 2002), total specific NFκB DNA-binding protein was determined by electrophoretic morbility shift assay (EMSA) as follows. A duplexed oligonucleotide probe (Promega, Madison, Wis.) with sense strand containing the decameric κB consensus binding sequence (underlined) 5′-AGTTGAGGGGACTTTCCCAGGC-3′(SEQ ID NO:4) was end-labeled with [γ-P32]ATP (PerkinElmer, Boston, Mass.) using T4 polynucleotide kinase (Promega, Madison, Wis.) and purified with G-50 Sephadex Bio-Spin columns (BioRad, Hercules, Calif.). The binding reaction was performed by preincubation on ice for 15 min with 10 μg nuclear protein and 5 μl binding buffer (50 mM Tris-HCl, pH 7.5; 5 mM MgCl2; 2.5 mM EDTA; 2.5 mM DTT; 250 mM NaCl; 20% glycerol; and 0.25 mg/ml poly[dI-dC]) before addition of the radiolabeled probe for an additional 20 min (room temperature). To confirm specific NFκB protein binding to the probe, supershift assays were performed using p50 and p65 subunit specific antibodies (Santa Cruz Biotechnology); nuclear extracts were preincubated with 1 μl of each antibody solution for 30 min at room temperature before addition of the radiolabeled oligonucleotide probes. DNA-protein complexes were resolved by electrophoresis using native 5% polyacrylamide gels run in 0.5×TBE (45 mM Tris-HCl; 45 mM boric acid; 1 mM EDTA) buffer. The gels were dried on filter papers at 80° C., and then exposed overnight to X-ray film at −80° C. using an intensifying screen.

Quantitative p50 and p65 NFκB DNA-binding were also determined using ELISA based Trans-AMTM assays in accordance with the manufacturer's instructions (ActiveMotif; Carlsbad, Calif.). In these commercial kits, a duplexed NFκB oligonucleotide containing the same κB consensus sequence described for the EMSA assay above is attached to the surface of 96-well plates. Activated NFκB in tumor or nuclear extracts that is first bound to the attached oligonucleotide is specifically and quantitatively detected by subsequent incubation with p50 or p65 specific antibody followed by an enzyme (HRP)-linked secondary for colorimetric (OD450 nm absorbance) scoring. Standard curves to establish the linear assay range for Trans-AMTM determinations of p50 and p65 NFκB DNA-binding activities were performed using graded amounts of control (IL-1 treated) HeLa cell nuclear extracts (0.625 to 10 μg/well) and for comparison with a panel of nuclear extracts from six human breast cancer cell lines (MCF-7, T47D, BT474, MCF-7/HER2, SkBr3, MDA-231). Relative to untreated MCF-7 cells, ER-positive/ErbB2-negative T47D cells showed virtually identical levels of p65 and p50 DNA binding activities. In contrast, the ER-positive/ErbB2-positive BT474 and MCF-7/HER2 cells showed significantly greater p50 (1.7-fold and 2.8-fold, respectively) and marginally greater p65 (1.3-fold) DNA-binding activities. The ER-negative/ErbB2-positive SkBr3 cells and the ER-negative/Ras-mutated MDA-231 cells showed the highest levels of p50 (3.9-fold and 4.2-fold, respectively) and p65 (1.4-fold and 3.0-fold, respectively) DNA binding activities within the breast cancer cell line panel.

Statistical Analyses.

For comparison of dichotomous biomarker parameters (e.g. present or absent Sp1 or NFκB DNA-binding) within or between tumor groups (e.g. tumor Group A and tumor Group B), chi-square contingency tables were used to test for levels of significance (p-values). Means, medians, and standard deviations were calculated for all continuous biomarker values. For comparisons between normally distributed biomarker values, Pearson correlation coefficients (r) were calculated; otherwise, Spearman rank correlation coefficients (rs) were determined. Correlations were shown on scatter plots and tested for significance by t-test. Associations between continuous biomarker values were also displayed as notch-boxplots and tested for significance by the non-parametric Wilcoxon statistical test (notched region within each box represents the Wilcoxon determined variability about the median, with the lines outside each box representing outlier values beyond the 99.5 percentile). Metastatic breast cancer relapse and disease-free survival (DFS) status were available for all Group B tumor patients. Biomarker associations with cancer relapse status and DFS were tested for significance by univariate Cox model analyses; and Kaplan-Meier DFS curves defined by regression tree-determined optimum cut-points were tested for significance by log rank analyses. Multivariate Cox modeling was used to look for factors independently associated with DFS. All p-value determinations are for two-sided testing.

Results

Oxidant Induction of NFκB DNA-Binding in ER-Positive Breast Cancer Cells is Revented by Inhibitors of NFκB Activation.

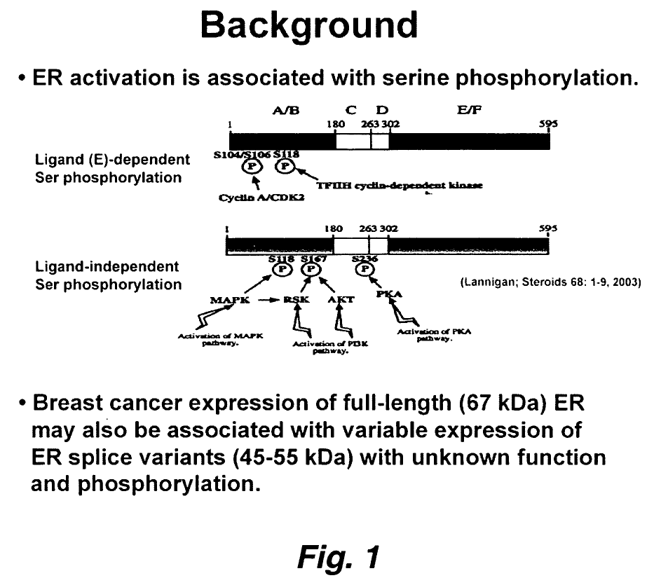

ER-positive breast cancer cells (MCF-7) were used to confirm that oxidant stress can activate and induce nuclear translocation of NFκB, leading to increased NFκB DNA-binding activity as measured from their nuclear extracts. The vitamin K analog, menadione, was chosen to induce intracellular oxidant stress as it is a widely employed model quinone that undergoes intracellular redoxcycling to generate excess reactive oxygen species (Bolton et al., 2000); it has also been used to demonstrate the role of oxidant stress in estrogen-induced carcinogenesis (Bhat et al., 2003). FIG. 1 shows the basal level of NFκB activation in ERpositive/ErbB2-negative MCF-7 cells relative to the constitutively increased level of NFκB activation present in ER-positive/ErbB2-positive MCF-7/HER2 and BT474 breast cancer cell lines (FIG. 1, panel A). Using specific antibodies to supershift the more intense EMSA complex produced by menadione treatment (100 μM×30 min) of MCF-7 cells confirmed that this complex contains both p50 and p65 subunits (FIG. 1, panel B). This EMSA demonstrated increase in MCF-7 NFκB activation following menadione treatment occurred in parallel with immunoblot-detected increases in the nuclear NFκB subunits, p50 and p65 (FIG. 1, panel D). As well, cytoplasmic levels of IκBα were reduced following menadione treatment, consistent with treatment enhanced proteasomal degradation of this NFκB inhibitor. In contrast, cytoplasmic IκBα levels were restored or even increased above control levels when menadione treated cells were pretreated with drugs known to inhibit intracellular NFκB activation by diverse intracellular mechanisms (FIG. 1, panel D). The proteasome inhibitors MG-132 and PS-341, the antioxidant PDTC, and the specific IκB kinase (IKK) inhibitor PA each 14 caused complete inhibition of NFκB DNA-binding (FIG. 1, panel C), along with marked reductions in nuclear p50 and p65 NFκB subunit content (FIG. 1, panel D).

NFκB Inhibition by PA Sensitizes ER-Positive/ErbB2-Positive Breast Cancer Cells to the Antiestrogen Tamoxifen.

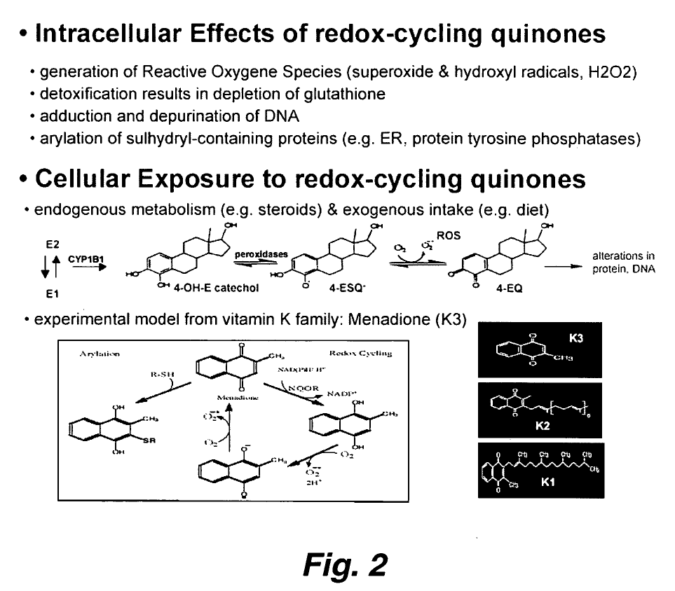

ER-positive MCF-7/HER2 and BT474 breast cancer cells differ from ER-positive MCF-7 breast cancer cells primarily by their marked overexpression of the oncogenic receptor tyrosine kinase, HER2/ErbB2, which is thought to diminish the sensitivity of ER-positive breast cancer cells to the antiproliferative activity of antiestrogens like tamoxifen (Benz et al., 1992; Benz, 2004). After 5-7 days treatment by tamoxifen at concentrations approaching 1000 nM, MCF-7 cells demonstrate nearly 50% reduction in viable cell number while cultured MCF-7/HER2 and BT474 cells show only ˜25% cellular reductions (Benz et al., 1992). The growth-inhibiting effects of tamoxifen are not generally apparent within 24 h of culture treatment, even for sensitive cell lines like MCF-7, unless tamoxifen is co-administered with an additively (or super-additively) active anticancer agent. As shown in FIG. 2, cotreatment of cell cultures with an NFκB-inhibiting dose of PA (50 μM) in combination with a standard dose of tamoxifen (500 nM) causes a significant and greater than expected reduction in cell viability at 24 h for the antiestrogen-resistant MCF-7/HER2 and BT474 cells, but not for the antiestrogen-sensitive MCF-7 cells. While this dose of PA effectively inhibited NFκB activation in all three of the ER-positive cell lines (FIG. 1; data not shown), this more selective sensitizing effect of PA on tamoxifen was only apparent against the ErbB2-positive MCF-7/HER2 and BT474 cells since, as noted earlier, ErbB2 overexpression is associated with increased NFκB activation (2-fold increased NFκB DNA-binding in MCF-7/HER2 vs. MCF-7 cells), consistent with mechanistic evidence 15 that this activation of NFκB is caused by HER2/ErbB2-induced IκBα degradation (Romieu-Mourez et al., 2002).

Breast Cancer NFκB Activity is Dependent on the Level of ER Expression.

Cryobanked extracts from two different groups of ER-positive primary breast cancers with markedly different ER content were analyzed to compare NFκB DNA-binding activities, since previous clinical studies had been restricted to ER-negative breast cancers and cell lines (Biswas et al., 2001; Nakshatri et al., 1997), and basic studies had indicated that ER and NFκB mutually inhibit the transcriptional activities of one another (Harnish et al., 2000; Rae et al., 1997; Speir et al., 2000). Within the composite collective of ER-positive breast cancer samples (Group A+Group B; n=81), DNAbinding complexes containing the p50 NFκB subunit were almost 2-fold more abundant than those containing the p65 NFκB subunit as shown in ure 3A; although activation of each of these two subunits, independently measured by the quantitative ELISA-based DNA-binding assays, appeared to be tightly correlated (rs=0.86; p<0.0001). Group B breast cancers with a median <0.5-fold ER content (range 21-87 fmol/mg; n=59) showed significantly higher NFκB DNA-binding than the Group A tumors with higher ER expression (>100 fmol/mg; n=22). As shown in ure 3B, this overall excess in NFκB activity observed in the Group B tumors represented a highly significant 2-fold increase in DNA-binding by the p50 NFκB subunit and a 4-fold increase in DNA-binding by the p65 NFκB subunit, over that measured in the Group A tumors (p<0.0001). Thus, ERpositive primary breast cancers can be subset according to both NFκB activity and ER content; tumors with increased ER content have significantly lower NFκB activity.

Correlations Between NFκB Activity and Other Breast Cancer Biomarkers.

In addition to ER content and NFκB DNA-binding, the only other biomarker measured in both Group A and B tumors was Sp1 DNA-binding, performed by EMSA as previously described (Quong et al., 2002). While a significant inverse correlation between NFκB DNA-binding and Sp1 DNA-binding has been reported for normal aging organs (Helenius et al., 1996), we did not detect any significant associations between these two redox-sensitive parameters either across the combined tumor set or within the two groups. Within Group A and Group B consistent trends were apparent in that cancer extracts showing complete loss of Sp1 DNA-binding contained up to 30% higher mean NFκB p50 DNA-binding activity, with an inverse correlation between p50 DNA-binding and Sp1 DNA-binding for the Group B samples of rs=-0.215 (p=0.29). Among these Group B tumors we had previously observed significant associations between loss of Sp1 DNA-binding, increased phospho-Erk5, and increasing patient age at diagnosis (Quong et al., 2002); however, no age association with NFκB p50 or p65 DNA-binding was apparent in either Group A or B tumors.

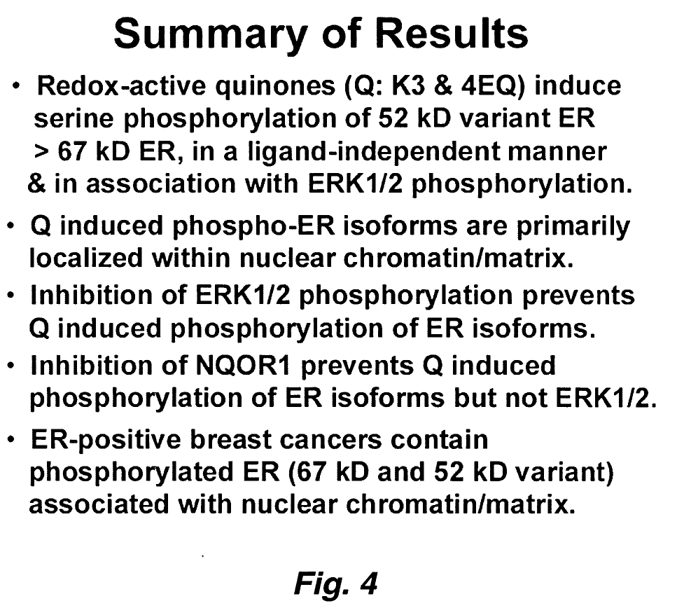

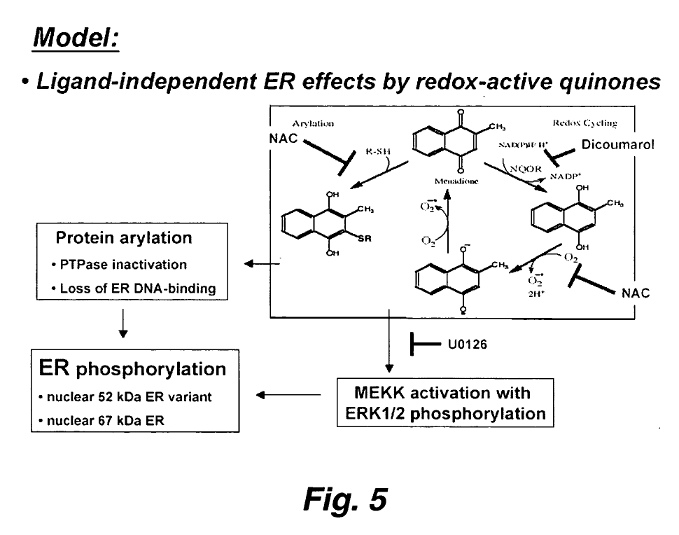

Since many of the Group B tumors had previously been analyzed for other biomarkers (Eppenberger et al., 1998; Eppenberger-Castori et al., 2001; Eppenberger-Castori et al., 2002; Quong et al., 2002), NFκB DNA-binding levels were correlated with all acquired biomarker data in addition to Sp1 DNA-binding. No significant correlations were observed between p50 or p65 NFκB DNA-binding and PR, pS2, phospho-Erk5, EGFR, iNOS, or cathepsin D expression. In contrast, significant positive correlations were observed between NFκB DNA-binding and ErbB2 expression, AP-1 DNA-binding, and uPA (FIGS. 4 and 5). The scatter plots in FIG. 4 show that DNA-binding by NFκB p50 (rs=+0.26; p=0.05) and p65 (rs=+0.29; p=0.03) subunits correlated comparably and significantly with total ErbB2 expression, but largely independent of the clinically validated threshold value (>500 U/mg) for ErbB2 overexpression previously linked with ErbB2 genomic amplification and worse patient prognosis (Eppenberger-Castori et al., 2001). The scatter plots in FIG. 5 show that DNA-binding by the NFκB p50 subunit correlated positively with both AP-1 DNA-binding (r=+0.34; p=0.01) and uPA expression (r=+0.43; p=0.008).

Increased NFκB p50 Activation Associates with Higher Risk of Breast Cancer Relapse and Reduced DFS.

Patient clinical follow-up data including metastatic breast cancer relapse and disease-free survival (DFS) status were available only for the Group B tumors. Box plots in FIG. 6 indicate that the primary breast cancers destined to relapse (13/59) possessed significantly higher NFκB p50 DNA-binding activity over those similarly staged ER-positive primary breast cancers not destined to relapse (46/59; p=0.04 by univariate Cox regression model). The generally lower NFκB p65 DNAbinding activities followed a similar trend but did not reach statistical significance. Independent regression tree analyses were performed to determine p50 and p65 DNAbinding value cutpoints (0.95 and 0.75, respectively) that would optimally separate Kaplan-Meier DFS curves for high vs. low NFκB subsets. FIG. 7 shows that higher NFκB p50 DNA-binding values were associated with significantly reduced DFS (p=0.04), in keeping with the p50 DNA-binding results shown in FIG. 6. Consistent with the p65 DNA-binding trend shown in FIG. 6, the Kaplan-Meier plots in FIG. 7 indicate that higher p65 DNA-binding were also associated with reduced DFS but this outcome difference did not reach statistical significance (p=0.09).

Dichotomizing the Group B tumor values for AP-1 and uPA in a similar fashion produced Kaplan-Meier DFS plots with even more significant outcome differences defined by these two biomarkers as compared to NFκB p50 DNA-binding (FIG. 7), although it should be noted that uPA values were not available on three of the Group B tumors and AP-1 DNA-binding values were not available on five of the Group B tumors. In a multivariate hazard Cox regression model in which all three parameters together predicted DFS (p=0.01; p65 DNA-binding excluded based on univariate analysis), only AP-1 and uPA showed independent prognostic significance (AP-1 DNA-binding p=0.06; uPA expression p=0.04).

Discussion

Constitutive upregulation of NFκB has been linked with the development and progression of ER-negative breast cancers in animal models and in humans (Cao and Karin, 2003); yet analyses of a limited number of ER-positive breast cancer models and tumor samples to date have suggested that NFκB may not play any clinical role in hormone-dependent breast tumorigenesis (Sovak et al., 1997; Nakshatri et al., 1997; Cogswell et al., 2000). To reassess this question, we studied three different ER-positive breast cancer cell models with known differences in their responsiveness to the clinically used antiestrogen, tamoxifen. Prior NFκB studies have focused on the ER-positive, tamoxifen-sensitive MCF-7 cells; in contrast, ER-positive MCF-7/HER2 and BT474 breast cancer cells differ from the MCF-7 breast cancer model primarily by their marked overexpression of the oncogenic receptor tyrosine kinase, HER2/ErbB2, which is known to activate NFκB expression (Romieu-Mourez et al., 2002; Biswas et al., 2004) and also diminish the responsiveness of ER-positive breast cancer cells to tamoxifen (Benz et al., 1992; Benz, 2004). The present study demonstrates that exogenous exposure to the ROS-generating quinone, menadione, can significantly increase MCF-7 NFκB activity and that this induction, associated with cytoplasmic loss of IκBα and nuclear translocation of both p50 and p65 NFκB subunits, is completely inhibited by a variety of NFκB-inhibiting drugs including the antioxidant PA, the proteasomal inhibitors MG-132 and PS-341, and the IKK inhibitor PA.

Additional studies with these breast cancer models were performed to explore the therapeutic potential of inhibiting NFκB as an adjunct to endocrine treatment for ERpositive breast cancers resistant to tamoxifen. As expected, endogenous levels of NFκB DNA-binding activity measured in the tamoxifen-resistant ER-positive/ErbB2-positive MCF-7/HER2 and BT474 cells were significantly increased above basal levels found in tamoxifen-sensitive ER-positive/ErbB-negative MCF-7 cells. Notably, the specific NF□B inhibitor PA given in combination with tamoxifen produced greater than additive reduction in MCF-7/HER2 and BT474 cell survival, but did not further sensitize MCF-7 cells to tamoxifen. These findings are also consistent with a recent report showing that inhibition of NF□B induction by co-treatment with PA overcomes the resistance to tamoxifen induced in an MCF-7 subline cells by constitutive overexpression of Akt (DeGraffenried et al., 2004).

The present study employed ELISA-based assays allowing for independent quantification of specific p65 and p50 DNA-binding subunits to address NFκB activation differences between breast cancer cell lines and concerns that breast cancer cell line models do not adequately reflect NFκB activation patterns observed in breast tumor samples (Cogsell et al., 2000). While NFκB DNA-binding activities measured in the ERpositive/ErbB2-positive MCF-7/HER2 and BT474 cell lines were significantly increased relative to that in ER-positive/ErbB2-negative MCF-7 cells, the ErbB2-associated induction of NFκB appeared primarily due to an increase in NFκB p50 subunit DNAbinding. Another ER-positive subline of MCF-7 (MCF-7/LCC1), previously generated by in vivo growth selection under estrogen withdrawal conditions and not by ErbB2 transduction, was recently shown to have a similar endogenous and selective increase in NFκB p50 subunit DNA-binding associated with upregulated Bcl-3 expression over basal levels measured in parental MCF-7 cells (Pratt et al., 2003). These findings suggest that cell line models representing different subsets of ER-positive breast cancers may show specific patterns of NFκB subunit activation; in particular, it is of interest that two antiestrogen-resistant ER-positive MCF-7 sublines generated independently and by different mechanisms (MCF-7/HER2, MCF-7/LCC1) resulted in similar selective increases in NFκB p50 subunit activation. Bcl-3 expression in the MCF21 7/HER2 subline has not yet been assessed; however, the observed upregulation of Bcl-3 expression along with p50 subunit activation in the MCF-7/LCC1 subline is consistent with the oncogenic ability of Bcl-3 to render p50 transcriptionally competent and stimulate NFkB-dependent target genes (Cogswell et al., 2000; Ghosh and Karin, 2002; Pratt et al., 2003).

To clarify the extent and importance of NFκB activation among biologically and clinically diverse sets of ER-positive primary breast tumors, specific p65 and p50 NFκB DNA-binding subunit activities were independently assessed in 81 samples grouped by high (Group A, n=22) vs. low (Group B, n=59) ER content. Over the entire collective p50 and p65 DNA-binding activities appeared tightly correlated (rs=0.86; p<0.0001); however, this very strong relationship also revealed a consistent ˜2-fold greater expression of p50 DNA-binding activity over p65 DNA-binding activity which was also apparent within each tumor group. Notable was the significant inverse relationship observed between p50 and p65 DNA-binding activities and tumor ER content; the lower ER expressing Group B tumors exhibited a mean 2-fold higher level of p50 DNA-binding activity and mean 4-fold higher p65 DNA-binding activity than the higher ER expressing Group A tumors (p<0.0001). While all ER-positive breast tumors are recommended for treatment with an endocrine agent like tamoxifen, breast tumors with lower ER content are known to have a lower likelihood of clinical response to endocrine therapy (Benz, 2004), suggesting that increased p50 and/or p65 NF□B activation accompanies the clinical development of higher-risk endocrine-resistant breast cancers. It remains to be determined if the mechanism accounting for this inverse correlation between breast cancer ER content and degree of NFκB activation relates to the potent repressive effect that nuclear p65 NFκB exerts on virtually all members of the steroid receptor family including ER (McKay and Cidlowski, 1998). This could potentially explain why the nuclear p65 subunits showed a greater magnitude inverse relationship to tumor ER content, despite the fact that the p50 NF□B subunits were more highly activated in these ER-positive breast cancers.

Across the entire collective and within individual tumor groups, we observed no significant associations between p50 or p65 NFκB DNA-binding activities and either patient age at diagnosis or change in another redox-sensitive breast tumor biomarker, Sp1 DNA-binding. NFκB DNA-binding and Sp1 DNA-binding have been reported to show inverse correlations with aging in normal mammalian organs (Helenius et al., 1996). This is of further interest since in the Group B tumors we had previously demonstrated a significant association between loss of Sp1 DNA-binding, increase in the oxidant-sensitive phospho-Erk5 biomarker, and older patient age at tumor diagnosis (Quong et al., 2002). Non-significant inverse correlations between p50 DNA-binding and loss of Sp1 DNA-binding were apparent in both tumor groups, suggesting either that this study was underpowered to adequately prove this inverse relationship or that the mechanism by which NF□B becomes activated during mammary gland tumorigenesis supercedes or is independent of those affecting other redox-sensitive biomarkers altered during aging and tumorigenesis.

Among Group B tumor samples for which additional biomarker and clinical followup data were available, NFκB p50 subunit activation correlated with other validated breast cancer prognostic biomarkers and with clinical outcome measures, moreso than did p65 subunit activation. The exception to this general pattern was the significant correlation observed between both p50 DNA-binding (rs=0.26; p=0.05) and p65 DNAbinding (rs=0.29; p=0.03) activities and tumor ErbB2 receptor content. Mechanistically, this correlation could have resulted either from the putative regulation of ErbB2 transcriptional expression by NFκB activation (Raziuddin et al., 1997), or by the well described signal activation of NFκB by oncogenic overexpression of the ErbB2 receptor tyrosine kinase (Romieu-Mourez et al., 2002; Biswas et al., 2004). Since only 8 (14%) of the 59 Group B tumors met the validated threshold criterion of >500 U/mg receptor protein for oncogenic ErbB2 overexpression (Eppenberger-Castori et al., 2001), and neither p50 nor p65 DNA-binding activities were significantly higher in these ErbB2 overexpressing tumors as compared to the lower ErbB2 expressing tumors, the functional and clinical significance of this correlation between NFκB activation and ErbB2 receptor content awaits further evaluation in a larger collection of ErbB2 overepressing breast cancers. The clinical importance of this observed correlation with ErbB2 content may also be questioned by the fact that in these Group B tumors, ErbB2 expression levels showed no significant association with any clinical outcome measure, yet the level of NFκB p50 subunit activation was significantly higher in breast tumors destined to relapse and predicted significantly for patient disease-free survival (DFS).

In this same group of histologically and morphologically homogenous breast cancers, we found that NF□B p50 subunit activation, but not p65 subunit activation, correlated significantly with two other biomarkers of clinical and mechanistic relevance to hormone-dependent breast cancer prognosis: AP-1 DNA-binding activity (r=0.34, p=0.01) and uPA expression (r=0.43; p=0.008). Intracellularly activated NFκB and AP-1 transcription factor complexes bind to their respective uPA promoter elements and cooperatively stimulate expression of this secreted protease known to drive tumor cell invasion and metastasis (Hansen et al., 1992; Sliva et al., 2002). While the observation that increased expression of uPA correlates strongly with increased NFκB p50 activation and AP-1 DNA-binding may be appealing from a mechanistic perspective, the failure of NFκB p65 DNA-binding to show comparable correlations is unexpected and may reflect either the limited number of samples studied (since similar but non-significant trends were observed for p65) or the preferential activation of NFκB p50 subunits in these breast tumor samples, as observed by others (Cogswell et al., 2000).

From earlier studies on different sample sets, we had demonstrated that increased uPA expression independently identifies a high-risk subset of node-negative breast cancers (Eppenberger et al., 1998), and that development of clinical resistance to tamoxifen in ER-positive tumors is associated with increased AP-1 DNA-binding activity (Johnston et al, 1999). Given that NFκB p50 activation correlates strongly with both increased AP-1 DNA-binding and uPA expression, it would be reasonable to suspect that increased NF□B p50 DNA-binding might also identify a high-risk subset of ERpositive (and tamoxifen resistant) breast cancers. From the clinical follow-up data available on the Group B tumors, NFκB p50 DNA-binding activity was found to be significantly increased in those primary breast tumors destined to relapse (13/59; p=0.04 by univariate Cox regression model analysis). While showing a similar trend, increased NFκB p65 DNA-binding was not significantly associated with relapsing tumors. Consistent with these NF□B differences in relapsing vs. non-relapsing breast tumors, Kaplan-Meier survival analyses confirmed that higher NFκB DNA-binding values were associated with reduced DFS outcomes. DFS differences reached significance for p50 DNA-binding (p=0.04); and although a similar DFS trend was apparent for p65 DNA binding it did not reach statistical significance (p=0.09). Kaplan-Meier analyses revealed even more striking DFS differences determined by AP-1 DNA-binding activities (p=0.009) and uPA expression levels (p=0.001), consistent with conclusions drawn from our earlier studies (Eppenberger et al., 1998; Johnston et al., 1999). In a multivariate analysis comparing NFκB p50 and p65 DNA-binding activities against AP-1 DNA-binding and uPA expression in determining DFS, only AP-1 and uPA demonstrated independent prognostic significance (AP-1 DNA-binding p=0.056; uPA expression p=0.039). These outcome assessments lend further support to the mechanistic hypothesis that the prognostic ability of increased NFκB DNA-binding to identify a high-risk subset of hormone-dependent breast cancers is mediated at least in part by its transcriptional stimulation of uPA expression, in concert with increased AP-1 DNA-binding.

In sum, this clinical outcome study is the first to demonstrate that ER-positive primary breast cancers can be prognostically subdivided according to NFκB activity, with increased p50 subunit DNA-binding activity appearing to be clinically more significant than increased p65 subunit DNA-binding activity. Our additional results comparing NF□B DNA-binding activities in selected ER-positive breast cancer cell line models are in keeping with other recent reports showing selective activation of NF□B p50 in association with reduced tamoxifen sensitivity, and provide rationale for further preclinical efforts aimed at evaluating the feasibility of therapeutically inhibiting NF□B activity in order to improve efficacy of antiestrogen treatment in patients with high-risk hormone-dependent breast cancer. NFκB

### REFERENCES

Allen and Tresini (2000). Oxidative stress and gene regulation. Free Rad. Biol. Med. 28: 463-499.

Baeuerle, P. A., and Baltimore, D. (1996). NF-κB: ten years after. Cell 87: 13-20.

Baldwin, A. S. (2001). Control of oncogenesis and cancer therapy resistance by the transcription factor NF-κB. J. Clin. Invest. 107: 241-246.

Benz, C C. (2004). ErbB2/HER2 and other molecular pathways in ER-positive breast cancer: impact on endocrine resistance and clinical outcome. In: M. Dowsett & J. Ingle (Eds.), Endocrine Therapy in Breast Cancer (in press). New York: Marcel Dekker, Inc.

Benz, C. C., Scott, G. K., Sarup, J. C., Johnson, R. M., Tripathy, D., Coronado, E., Shepard, H. M., and Osborne, C. K. (1992). Estrogen-dependent tamoxifen-resistant tumorigenic growth of MCF-7 cells transfected with HER2/neu. Breast Cancer Res. Treat. 24: 85-95.

Bhat, H. K., Calaf, G., Hei, T. K., Loya, T., and Vadgama, J. V. (2003). Critical role of oxidative stress in estrogen-induced carcinogenesis. Proc. Natl. Acad. Sci. (USA) 100: 3913-3918.

Biswas, D. K., Dai, S-C., Cruz, A., Weiser, B., Graner, E., and Pardee, A. B. (2001). The nuclear factor kappa B (NF-κB): a potential therapeutic target for estrogen receptor negative breast cancers. Proc. Natl. Acad. Sci. (USA) 98:10386-10391.

Biswas, D. K., Shi, Q., Baily, S., Strickland, I., Ghosh, S., Pardee, Å. B., and Iglehart, J. D. (2004). NF-kappaB activation in human breast cancer specimens and its role in cell proliferation and apoptosis. Proc. Natl. Acad. Sci. (USA) 101: 10137-10142.

Bolton, J. L., Trush, M. A., Penning, T. M., Dryhurst, G., and Monks, T. J. (2000). Role of quinones in toxicology. Chemical Research in Toxicology 13: 135-160.

Cao, Y., and Karin, M. (2003). NF-κB in mammary gland development and breast cancer. J. Mammary Gland Biology and Neoplasia 8: 215-223.

Cogswell, P. C., Guttridge, D. C., Funkhouser, W. K., and Baldwin, A. S. Jr. (2000). Selective activation of NF-κB subunits in human breast cancer: potential roles for NF-κB2/p52 and for Bcl-3. Oncogene 19: 1123-1131.

DeGraffenried, L. A., Chandrasekar, B., Friedrichs, W. E., Donzis, E., Silva, J., Hidalgo, M., Freeman, J. W., and Weiss, G. R. (2004). NF-kappaB inhibition markedly enhances sensitivity of resistant breast cancer tumor cells to tamoxifen. Ann. Oncol. 5: 885-890.

Dignam, J. D., Martin, P. L., Shastry, B. S., and Roeder, R. G. (1983). Eukaryotic gene transcription with purified components. Methods Enzymol. 101: 582-598.

Dixit, V., and Mak, T. W. (2002). NF-κB signaling: many roads lead to Madrid. Cell 111: 615-619.

Eppenberger, U., Kueng, W., Schlaeppi, J-M., Roesel, J. L., Benz, C., Mueller, H., Matter, A., Zuber, M., Leuscher, K., Litschgi, M., Schmitt, M., Foekens, J. A., and Eppenberger-Castori, S. (1998). Makers of tumor angiogenesis and proteolysis independently define high- and low-risk subsets of node-negative breast cancer patients. J. Clin. Oncol. 16:3129-3136.

Eppenberger-Castori, S., Kueng, W. Benz, C. C., Paris, K., Caduff, R., Bannwart, F., Fink, D., Dieterich, H., Braschler, C., von Castelberg, B., Muller, H., and Eppenberger, U. (2001). Prognostic and predictive significance of ErbB2 breast tumor levels measured by enzyme-immuno-assay (EIA). J. Clin. Oncol. 19: 645-656.

Eppenberger-Castori, S. Moore II, D., Thor, A. D., Edgerton, S. M., Kueng, W., Eppenberger, U., and Benz, C. C. (2002). Age-associated biomarker profiles of human breast cancer. Int. J. Biochem. & Cell Biol. 34: 1318-1330.

Feinman, R., Siegel, D. S., and Berenson, J. (2004). Regulation of NF-κB in multiple myeloma: therapeutic implications. Clin. Adv. Hematol. Oncol. 2: 162-166.

Giardina, C., and Hubbard, A. K. (2002). Growing old with nuclear factor-kappaB. Cell Stress Chaperones 7: 207-212.

Ghosh, S., and Karin, M. (2002). Missing pieces in the NF-κB puzzle. Cell 109: S81-S96.

Ghosh, S., May, M. J., and Kopp, E. B. (1998). NF-kappaB and Rel proteins: evolutionarily conserved mediators of immune response. Annu. Rev. Immunol. 16: 225-260.

Hansen, S. K., Nerlov, C., Zabel, U., Verde, P., Johnsen, M., Baeuerle, P. A., and Blasi. F. (1992). A novel complex between the p65 subunit of NF-κB and c-Rel binds to a DNA element involved in the phorbol ester induction of the human urokinase gene. EMBO J. 11: 205-213.

Harnish, D. C., Scicchitano, M. S., Adelman, S. J., Lyttle, C. R., Karathanasis, S. K. (2000). The role of CBP in estrogen receptor cross-talk with nuclear factor-kappaB in HepG2 cells. Endocrinology 141: 3403-3411.

Hehner, S. P., Hofmann, T. G., Droge, W., and Schmitz, M. L. (1999). The anti-inflammatory sesquiterpene lactone parthenolide inhibits NF-κB by targeting the IκB kinase complex. J. Immunol. 163: 5617-5623.

Helenius, M., Hanninen, M., Lehtinen, S. K., and Salminen, A. (1996). Aging-induced upregulation of nuclear binding activities of oxidative stress responsive NF-κB transcription factor in mouse cardiac muscle. J. Mol. Cell. Cardiol. 28: 487-498.

Johnston, S. R. D., Lu, B., Scott, G. K., Kushner, P. J., Smith, I. E., Dowsett, M., and Benz, C. C. (1999). Increased activator protein-1 DNA binding and c-Jun NH2-terminal kinase activity in human tumors with acquired tamoxifen resistance. Clin. Cancer Res. 5:251-256, 1999.

Karin, M., and Lin, A. (2002) NF-κB at the crossroads of life and death. Nature Immunol. 3: 221-227.

Karin, M. Yamamoto, Y., and Wang, Q. M. (2004). The IKK NF-kappaB system: a treasure trove for drug development. Nat. Rev. Drug Discov. 3: 17-26.

Liang, X., Lu, B., Scott, G. K., Chang, C.-H., Baldwin, M. A., and Benz, C. C. (1998). Oxidant stress impaired DNA-binding of estrogen receptor from human breast cancer. Mol. Cell. Endocrinol. 146: 151-161.

McKay, L. I., and Cidlowski, J. A. (1998). Cross-talk between nuclear factor-κB and the steroid hormone receptors: mechanisms of mutual antagonism. Mol. Endocrinol. 12: 45-56.

Nakshatri, H., Bhat-Nakshatri, P., Martin, D. A., Goulet, Jr., R. J., and Sledge, Jr., G. W. (1997). Constitutive activation of NF-κB during progression of breast cancer to hormoneindependent growth. Mol. Cell. Biol. 17:3629-3639.

Pahl, H. L. (1999). Activators and target genes of Rel/NF-kappaB transcription factors. Oncogene 18: 6853-6866.

Quong, J., Eppenberger-Castori, S., Moore III, D., Scott, G. K., Birrer, M. J., Kueng, W., Eppenberger, U., and Benz, C. C. (2002). Age-dependent changes in breast cancer hormone receptors and oxidant stress markers. Breast Cancer Res. Treat. 76:221-236.

Ray, P., Ghosh, S. K., Zhang, D. H., and Ray, A. (1997). Repression of interleukin-6 gene expression by 17β-estradiol: inhibition of the DNA-binding activity of the transcription factors NF-IL6 and NF-kappaB by the estrogen receptor. FEBS Lett. 409: 79-85.

Radiuddin, A., Court, D., Sarkar, F. H., Liu, Y.-L., Kung, H.-F., and Radiuddin, R. (1997). A c-erbB-2 promoter-specific nuclear matrix protein from human breast tumor tissues mediates NF-κB DNA binding activity. J. Biol. Chem. 272: 15715-15720.

Romieu-Mourez, R., Landesman-Bollag, E., Seldin, D. C., and Sonnenshein, G. E. (2002). Protein kinase CK2 promotes aberrant activation of nuclear factor-kappaB, transformed phenotype, and survival of breast cancer cells. Cancer Res. 62: 6770-6778.

Schreck, R., Meier, B., Mannel, D. N., Droge, W., and Baeuerle, P. A. (1992). Dithiocarbamates as potent inhibitors of nuclear factor kappa B activation in intact cells. J. Exp. Med. 175: 1181-1194.

Skehan, P., Storeng, R., Scudiero, D., Monks, A., McMahon, J., Vistica, D., Warren, J. T., Bokesch, H., Kenney, S., and Boyd, M. R. (1990). New colorimetric cytotoxicity assay for anticancer-drug screening. J. Natl. Cancer Inst. 82: 1107-1112.

Sliva, D., English, D., Lyons D., and Lloyd Jr., F. P. (2002). Protein kinase C induces motility of breast cancers by upregulating secretion of urokinase-type plasminogen activator through activation of AP-1 and NF-κB. Biochem. Biophys. Res. Commun. 290: 552-557.

Sovak, M. A., Bellas, R. E., Kim, D. W., Zanieski, G. J., Rogers, A. E., Traish, A. M., and Sonenshein, G. E. (1997). Aberrant nuclear factor-κB/Rel expression and the pathogenesis of breast cancer. J. Clin. Invest. 100: 2952-2960.

Speir, E., Yu, Z. X., Takeda, K., Ferrans, V. J., and Cannon, R. O. (2000). Competition for p300 regulates transcription by estrogen receptors and nuclear factor-kappaB in human coronary smooth muscle cells. Circ. Res. 87: 1006-1011.

Veiby, O. P., and Read, M. A. (2004). Chemoresistance: impact of nuclear factor (NF)-κB inhibiton by small interfering RNA. Clin. Cancer Res. 10: 3262-3264.

Yamamoto, Y., and Gaynor, R. B. (2001). Therapeutic potential of inhibiton of the NF-κB pathway in the treatment of inflammation and cancer. J. Clin. Invest. 107: 135-142.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of identifying cancer patients less likely to respond to hormonal therapy, said method comprising:
obtaining a biological sample from a cancer patient wherein said biological sample comprises cancer cells; and
determining NF-κB levels, activity or DNA binding wherein a patient having higher NFκB levels, activity or DNA binding, as compared to the NF-κB levels, activity or DNA binding found in a normal healthy subject indicates that said patient is less likely to respond to hormonal therapy.

2. The method of claim 1, wherein said cancer patient is a cancer patient having ER positive breast cancer.

3. The method of claim 1, wherein said determining comprises determining NF-κB DNA binding.

4. The method of claim 1, wherein said determining comprises determining NF-κB activation.

5. A method of evaluating the prognosis of a patient having breast cancer, said method comprising:
obtaining a biological sample from said cancer patient wherein said biological sample comprises cancer cells; and
determining NFκB levels, activity or DNA binding in said cancer cells wherein higher NFκB levels, activity or DNA binding, as compared to the levels, activity, or DNA binding found in a normal healthy subject is an indicator of a higher risk of cancer recurrence or relapse.

6. The method of claim 5, wherein said cancer patient is a cancer patient having ER positive breast cancer.

7. A method of mitigating one or more symptoms of breast cancer in a subject having ER-positive breast cancer said method comprising administering to said patient a NF-κB inhibitor.

8. The method of claim 7, wherein said inhibitor inhibits NF-κB expression.

9. The method of claim 7, wherein said inhibitor inhibits DNA binding by NF-κB.

10. The method of claim 7, wherein said inhibitor is selected from an the group consisting of an inhibitor listed in Table 1, an inhibitor listed in Table 2, an inhibitor listed in Table 3, and an inhibitor listed in Table 4.

11. A method of identifying ligand-independent activation an estrogen receptor in a cell, said method comprising:
detecting the 52 kDa variant of the estrogen receptor in the nucleus of said cell; wherein increase levels of said 52 kDa variant in the nucleus of said cell as compared to that found in a cell that is not undergoing ligand-independent activation of the estrogen receptor indicates that ligand-independent activation of an estrogen receptor is occurring in said cell.

12. The method of claim 11, wherein said detecting comprises detecting the amount of 52 kDa variant in the nucleus.

13. The method of claim 11, wherein said detecting comprises detecting the amount of phosphorylated 52 kDa variant in said cell.

14. The method of claim 13, wherein said detecting comprises detecting the ratio of phosphorylated to unphosphorylated 52 kDa variant in said cell.

15. A method of selecting a therapeutic regimen for treatment of a cancer in a subject, said method comprising:
providing a biological sample from said subject comprising cancer cells;
detecting the 52 kDa variant of the estrogen receptor in the nucleus of the cancer cells; wherein increase levels of said 52 kDa variant in the nucleus of the cells as compared to that found in a cell that is not undergoing ligand-independent activation of the estrogen receptor indicates that said subject is a candidate for treatment of a cancer mediated by ligand-independent activation of the estrogen receptor.

16. The method of claim 15, wherein said detecting comprises detecting the amount of phosphorylated 52 kDa variant in said cell.

17. The method of claim 15, wherein said detecting comprises detecting the ratio of phosphorylated to unphosphorylated 52 kDa variant in said cell.

18. The method of claim 15, further comprising treating those candidates for treatment of a cancer mediated by ligand-independent activation of the estrogen receptor comprising by inhibiting a NQOR1 pathway and/or a MAPK pathway in cells comprising said cancer.

19. A kit for mitigating ligand independent activation of a nuclear steroid receptor, said kit comprising:
a container containing a MAPK inhibitor and/or an inhibitor of a vitamin K cycle; and
instructional materials teaching the use of a MAPK inhibitor and/or a quinine inhibitor for reducing ligand-independent activation of a nuclear steroid receptor.

20. A kit for identifying ligand-independent activation of an estrogen receptor, said kit comprising:
one or more reagents for detecting the amount and/or phosphorylation of the 52 kDa variant of the estrogen receptor in the nucleus of a cell.

21. The kit of claim 20, further comprising instructional materials teaching the detection of the amount and/or phosphorylation of said 52 kDa variant in the nucleus of a cell as an indicator of ligand-independent activation of said estrogen receptor.

22. A method of mitigating one or more symptoms of breast cancer said method comprising:
identifying a breast cancer patient wherein said breast cancer is an ER-positive breast cancers with elevated NFkB activity; and
administering, one or more NFkB inhibitors in conjunction with an antiestrogen.

23. The method of claim 22, wherein said NFκB inhibitor is parthenolide, or a parthenolide analogues.

24. The method of claim 22, wherein said antiestrogen is tamoxifen or 2-(4-Hydroxy-phenyl)-3-methyl-1-[4-(2-piperidin-1-yl-ethoxy)-benzyl]-1H-indol-5-ol hydrochloride (ERA-923).

25. The method of claim 22, wherein said NFκB inhibitor is administered before said antiestrogen.

26. The method of claim 22, wherein said NFκB inhibitor is administered after said antiestrogen.

27. The method of claim 22, wherein said NFκB inhibitor is administered with said antiestrogen.

28. A composition for mitigating one or more symptoms of breast cancer, said composition comprising an NFκB inhibitor combined with an antiestrogen.

29. The composition of claim 28, wherein said composition is formulated in a unit dosage formulation.

30. A kit for mitigating one or more symptoms of breast cancer, said kit comprising: an NFκB inhibitor and an antiestrogen.


SEQUENCE LISTING (4 sequences)

SEQ ID NO: 1 - DNA, 21 bp, Artificial
  Synthetic oligonucleotide primer.
ctcatgatcaaacgctctaag

SEQ ID NO: 2 - DNA, 20 bp, Artificial
  Synthetic oligonucleotide primer.
acggctagtgggcgcatgta

SEQ ID NO: 3 - DNA, 23 bp, Artificial
  Synthetic oligonucleotide primer.
catcaggtggatcaaagtgtctg

SEQ ID NO: 4 - DNA, 22 bp, Artificial
  Synthetic oligonucleotide primer.
agttgaggggactttcccaggc